(19) United States
(12) Patent Application Publication
(10) Pub. No.: US 2024/0132478 A1
(43) Pub. Date: Apr. 25, 2024

(54) COCRYSTAL

(71) Applicant: Syndesi Therapeutics SA, North Chicago, IL (US)

(72) Inventors: Andrew BAXTER, Sussex (GB); Suzanne BUTTAR, Cambridge (GB); Dyanne CRUIKSHANK, Cambridge (GB); Adam PATTERSON, Cambridge (GB)

(21) Appl. No.: 18/251,447

(22) PCT Filed: Nov. 1, 2021
(86) PCT No.: PCT/EP2021/080256

§ 371 Date: May 2, 2023

(30) Foreign Application Priority Data
Nov. 2, 2020 (EP) 20205195.9

(51) Int. Cl. C07D 403/06; C07C 65/03; C07C 65/05

(52) U.S. Cl. CPC C07D 403/06; C07C 65/03; C07C 65/05; C07B 2200/13

(57) ABSTRACT

The invention relates to cocrystals comprising a substituted 2-oxo-1-pyrrolidinyl triazole of formula (I) and a carboxylic acid of formula (II).

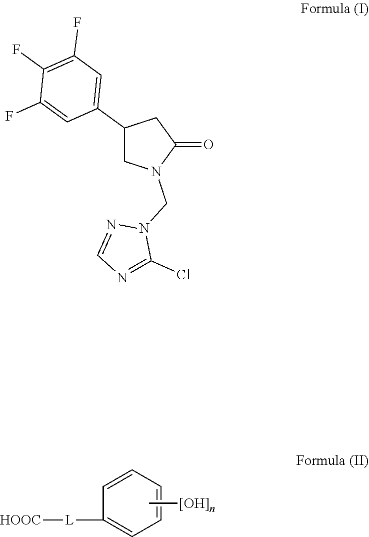

### FIELD OF THE INVENTION

The invention relates to cocrystals of the substituted 2-oxo-1-pyrrolidinyl triazole compound of formula (I).

### BACKGROUND

The substituted 2-oxo-1-pyrrolidinyl triazole compound of formula (I)

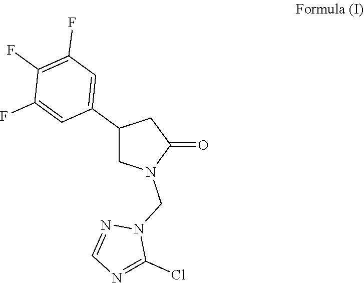 [SMILES: O=C1CC(c2cc(F)c(F)c(F)c2)CN1Cn1ncnc1Cl]

is a potential candidate for the treatment of cognitive impairment disorders such as Alzheimer's. A single form was observed in a previous polymorphism study. However, the melting point of that form is quite low at approximately 85° C. This may make particle-size reduction and formulation more challenging. The substituted 2-oxo-1-pyrrolidinyl triazole compound of formula (I) does not form simple salts, as the nitrogen atoms present are not easily protonated, so the option of forming a straightforward crystalline salt of the compound is not available.

There is therefore a need to develop new forms of the substituted 2-oxo-1-pyrrolidinyl triazole compound of formula (I) which are stable and exhibit useful properties that will aid the processing and formulation of a pharmaceutical product.

### SUMMARY OF THE INVENTION

The present invention provides cocrystals of the substituted 2-oxo-1-pyrrolidinyl triazole compound of formula (I) with improved physical properties compared to the pure compound.

It can be challenging to identify coformers that form a cocrystal with the API in question which provide an improvement in properties. The inventors have surprisingly found that the substituted 2-oxo-1-pyrrolidinyl triazole compound of formula (I) forms such cocrystals with a coformer which is a carboxylic acid of formula (II). What is more, the cocrystals of the present invention have a higher melting point than that of the pure API. Indeed, particular cocrystals of the invention exhibit melting points over 25° C. higher than that of the API. The higher melting point facilitates particle size reduction (e.g. micronization/nano milling) and subsequent downstream processing (e.g. dry granulation) by improving the crystallinity and powder flow properties of the API. The cocrystals of the present invention are also non-hygroscopic, stable over long periods at elevated temperature and humidity and have a high purity, and are predicted to withstand processing and storage conditions for solid pharmaceutical compositions.

The invention therefore provides a cocrystal comprising a substituted 2-oxo-1-pyrrolidinyl triazole compound of formula (I):

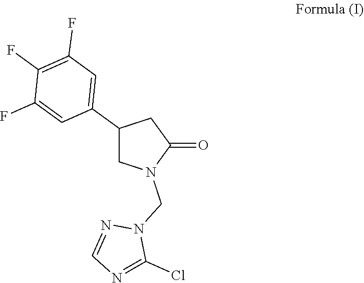 [SMILES: O=C1CC(c2cc(F)c(F)c(F)c2)CN1Cn1ncnc1Cl]

or an isomer thereof, and a carboxylic acid of formula (II):

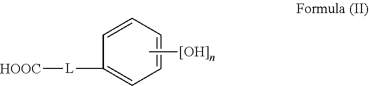

wherein n is from 1 to 5, and wherein L represents a bond or a C₁₋₄ alkylene group which is unsubstituted or substituted with one, two or three substituents selected from NH₂, OH, SH, fluorine or COOH.

The invention also provides a pharmaceutical composition comprising a cocrystal, the cocrystal comprising a substituted 2-oxo-1-pyrrolidinyl triazole compound of formula (I):

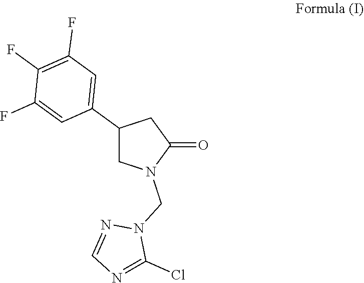 [SMILES: O=C1CC(c2cc(F)c(F)c(F)c2)CN1Cn1ncnc1Cl]

or an isomer thereof, and a carboxylic acid of formula (II):

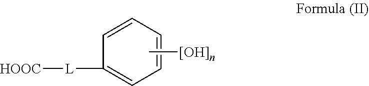

wherein n is from 1 to 5, and wherein L represents a bond or a C₁₋₄ alkylene group which is unsubstituted or substituted with one, two or three substituents selected from NH₂, OH, SH, fluorine or COOH.

The invention also provides a cocrystal comprising a substituted 2-oxo-1-pyrrolidinyl triazole compound of formula (I):

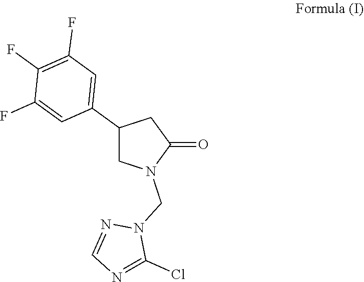 [SMILES: O=C1CC(c2cc(F)c(F)c(F)c2)CN1Cn1ncnc1Cl]

or an isomer thereof, and a carboxylic acid of formula (II):

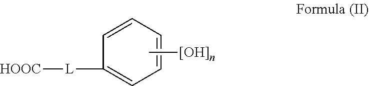

wherein n is from 1 to 5, and wherein L represents a bond or a C₁₋₄ alkylene group which is unsubstituted or substituted with one, two or three substituents selected from NH₂, OH, SH, fluorine or COOH for use in treating a cognitive disorder.

The invention also provides a method of treating a cognitive disorder in a subject, the method comprising administering a cocrystal which comprises a substituted 2-oxo-1-pyrrolidinyl triazole compound of formula (I):

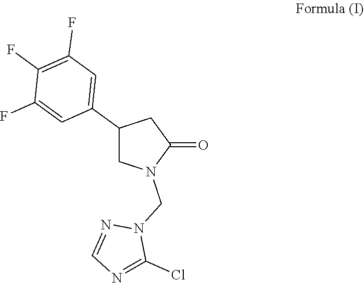 [SMILES: O=C1CC(c2cc(F)c(F)c(F)c2)CN1Cn1ncnc1Cl]

or an isomer thereof, and a carboxylic acid of formula (II):

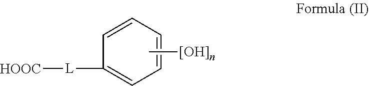

wherein n is from 1 to 5, and wherein L represents a bond or a C₁₋₄ alkylene group which is unsubstituted or substituted with one, two or three substituents selected from NH₂, OH, SH, fluorine or COOH.

The invention also provides the use of a cocrystal which is a cocrystal comprising a substituted 2-oxo-1-pyrrolidinyl triazole compound of formula (I):

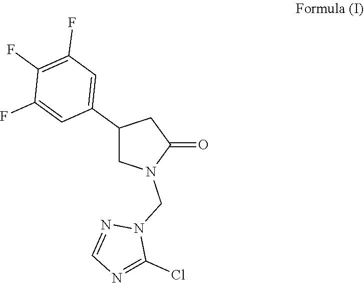 [SMILES: O=C1CC(c2cc(F)c(F)c(F)c2)CN1Cn1ncnc1Cl]

or an isomer thereof, and a carboxylic acid of formula (II):

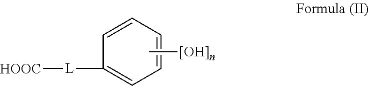

wherein n is from 1 to 5, and

wherein L represents a bond or a C₁₋₄ alkylene group which is unsubstituted or substituted with one, two or three substituents selected from NH₂, OH, SH, fluorine or COOH in the manufacture of a medicament for the treatment of a cognitive disorder

### BRIEF DESCRIPTION OF THE FIGURES

FIG. 1 shows the X-ray powder diffraction (XRPD) patterns for the scale up sample for 4-HBA with API (Pattern 1).

FIG. 2 shows the XRPD overlay of 4-HBA Pattern 1 before (ARP-1725-77-01A) and after (ARP-1725-77-O1B) drying in a vacuum oven.

FIG. 3 shows the XRPD patterns for the scale up sample for 2,4-DHBA with API (Pattern 1).

FIG. 4 shows the XRPD patterns for the scale up sample for 3,5-DHBA with API (Pattern 1).

FIG. 5 shows the XRPD patterns for samples ARP-1725-16-09_C and ARP-1725-16-03_C for the screen with L-tyrosine. The region highlighted in the black rectangle exhibits new reflections in the XRPD spectra not observed for API or conformer.

FIG. 6 shows a ball and stick representation of the 4-HBA cocrystal.

FIG. 7 shows the hydrogen bonding network of the 4-HBA cocrystal.

FIG. 8 shows a ball and stick representation of the 2,4-DHBA cocrystal.

FIG. 9 shows the hydrogen bonding network of the 2,4-DHBA cocrystal.

FIG. 10 shows a ball and stick representation of the 3,5-DHBA cocrystal.

FIG. 11 shows the hydrogen bonding network of the 3,5-DHBA cocrystal.

### DETAILED DESCRIPTION

The invention relates to a cocrystal comprising a substituted 2-oxo-1-pyrrolidinyl triazole compound of formula (I):

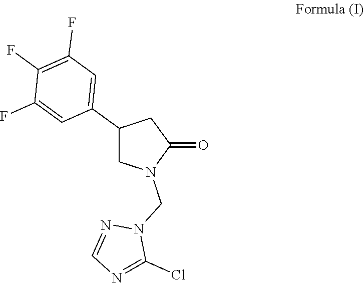 [SMILES: O=C1CC(c2cc(F)c(F)c(F)c2)CN1Cn1ncnc1Cl]

or an isomer thereof, and a coformer which is a carboxylic acid of formula (II):

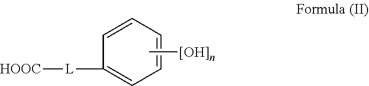

wherein n is from 1 to 5, and wherein L represents a bond or a C₁₋₄ alkylene group which is unsubstituted or substituted with one, two or three substituents selected from NH₂, OH, SH, fluorine or COOH.

The term “cocrystal” refers to a crystalline single-phase material comprising at least two components which is not a solvate or a simple salt. In the field of pharmaceuticals, the cocrystal typically comprises an API and a coformer.

The term “coformer” refers to a component in a cocrystal which is not the API, but is required for the cocrystal to form. The intermolecular interactions between the API and the coformer lower the energy of the system and result in stable crystalline solids which may have improved physical properties compared to API alone.

The term “crystalline” refers to a crystalline compound, which is a compound having an extended 3D crystal structure.

Reference to the substituted 2-oxo-1-pyrrolidinyl triazole compound of formula (I) as used herein includes any isomer thereof. In the context of the present invention, the term “isomer” refers to any geometric, optical, enantiomeric, diastereomeric, epimeric, atropic, stereoisomeric, tautomeric, conformational, or anomeric form of the compound. Preferably, the term isomer refers to stereoisomers. Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a substituted 2-oxo-1-pyrrolidinyl triazole of formula (I) refers to a compound having the same covalent linkages between atoms as depicted in formula (I).

The above exclusion does not pertain to tautomeric forms, for example, keto, enol, and enolate forms, as in, for example, the following tautomeric pairs: keto/enol, imine/enamine and amide/imino alcohol.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; F may be in any isotopic form, including ¹⁸F or ¹⁹F; and the like, unless otherwise specified.

The compound of formula (I) has an asymmetric centre, therefore the term “isomers” embraces all enantiomers of the compound of formula (I). The invention is to be understood to extend to the use of all such enantiomers, including such enantiomers alone and mixtures of such enantiomers in any proportion, including racemates. Formula (I) is intended to represent all individual stereoisomers and all possible mixtures thereof, unless stated or shown otherwise. In addition, compounds of formula (I) may exist as tautomers, for example keto (CH₂C═O)↔enol (CH═CHOH) tautomers or amide (NHC═O)↔hydroxyimine (N═COH) tautomers. Formula (I) is intended to represent all individual tautomers and all possible mixtures thereof, unless stated or shown otherwise.

Preferably, the term isomer refers to stereoisomers. Typically, the compound of formula (I) is a single stereoisomer or a mixture of stereoisomers.

Preferably, the compound of formula (I) comprises a compound of formula (Ia):

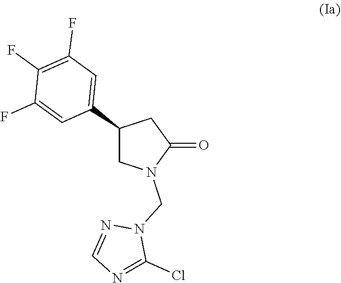 [SMILES: O=C1C[C@H](c2cc(F)c(F)c(F)c2)CN1Cn1ncnc1Cl]

For instance, the compound of formula (I) may comprise at least 50% of the compound of formula (Ia), at least 60% of the compound of formula (Ia), at least 70% of the compound of formula (Ia), at least 80% of the compound of formula (Ia), at least 90% of the compound of formula (Ia), at least 95% of the compound of formula (Ia), or at least 99% of the compound of formula (Ia).

Preferably, the compound is a substituted 2-oxo-1-pyrrolidinyl triazole of formula (Ia):

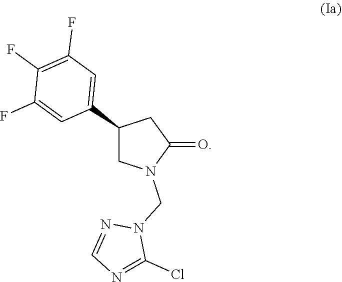 [SMILES: O=C1C[C@H](c2cc(F)c(F)c(F)c2)CN1Cn1ncnc1Cl]

Thus, the compound may comprise (4R)-1-[(5-chloro-1H-1,2,4-triazol-1-yl)methyl]-4-(3,4,5-trifluorophenyl)pyrrolidin-2-one. Preferably, the compound of formula (I) comprises at least 50% (4R)-1-[(5-chloro-1H-1,2,4-triazol-1-yl)methyl]-4-(3,4,5-trifluorophenyl)pyrrolidin-2-one, at least 60% (4R)-1-[(5-chloro-1H-1,2,4-triazol-1-yl)methyl]-4-(3,4,5-trifluorophenyl)pyrrolidin-2-one, at least 70% (4R)-1-[(5-chloro-1H-1,2,4-triazol-1-yl)methyl]-4-(3,4,5-trifluorophenyl)pyrrolidin-2-one, at least 80% (4R)-1-[(5-chloro-1H-1,2,4-triazol-1-yl)methyl]-4-(3,4,5-trifluorophenyl)pyrrolidin-2-one, at least 90% (4R)-1-[(5-chloro-1H-1,2,4-triazol-1-yl)methyl]-4-(3,4,5-trifluorophenyl)pyrrolidin-2-one, at least 95% (4R)-1-[(5-chloro-1H-1,2,4-triazol-1-yl)methyl]-4-(3,4,5-trifluorophenyl)pyrrolidin-2-one, or at least 99% (4R)-1-[(5-chloro-1H-1,2,4-triazol-1-yl)methyl]-4-(3,4,5-trifluorophenyl)pyrrolidin-2-one.

Preferably, the compound of formula (I) is (4R)-1-[(5-chloro-1H-1,2,4-triazol-1-yl)methyl]-4-(3,4,5-trifluorophenyl)pyrrolidin-2-one.

The coformer is a carboxylic acid of formula (II):

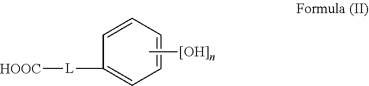

wherein n is from 1 to 5, and wherein L represents a bond or a C₁₋₄ alkylene group which is unsubstituted or substituted with one, two or three substituents selected from NH₂, OH, SH, fluorine or COOH.

As used herein, a C₁₋₄ alkylene group is an unsubstituted or substituted bidentate moiety obtained by removing two hydrogen atoms from a C₁ to C₄ alkane. The hydrogen atoms may be removed from the same carbon atom or from different carbon atoms. Examples of C₁ to C₄ alkylene groups include methylene, ethylene, propylene and butylene.

An alkylene group as used herein may be unsubstituted or substituted. Unless otherwise stated, substituted alkylene groups may carry one or more, e.g. one, two or three substituents. The substituents are selected from amine (—NH₂), hydroxyl (—OH), thiol (—SH), fluorine (—F) and carboxylic acid (—COOH). Preferred substituents are fluorine (—F), hydroxyl (—OH) groups and amine (—NH₂) groups, most preferably amine (—NH₂) groups. Where more than one substituent is present, these may be the same or different.

Typically n is from 1 to 4. For instance n may be from 1 to 3. Preferably n is 1 or 2.

In one aspect, L represents a bond. Thus, the coformer may be a carboxylic acid of formula (IIA):

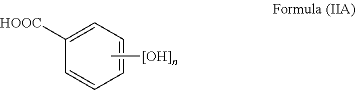

wherein n is from 1 to 5, for instance from 1 to 4, preferably from 1 to 3, more preferably 1 or 2.

Hence, the carboxylic acid of formula (II) or formula (IIA) may be a hydroxybenzoic acid, a dihydroxybenzoic acid or a trihydroxybenzoic acid. Preferably the carboxylic acid of formula (II) or formula (IIA) is a hydroxybenzoic acid or a dihydroxybenzoic acid.

Thus, the carboxylic acid of formula (II) or formula (IIA) may be selected from the group consisting of 2-hydroxybenzoic acid, 3-hydroxybenzoic acid, 4-hydroxybenzoic acid, 2,3-dihydroxybenzoic acid, 2,4-dihydroxybenzoic acid or 3,5-dihydroxybenzoic acid. Preferably, the carboxylic acid of formula (II) or formula (IIA) is selected from the group consisting of 4-hydroxybenzoic acid, 2,4-dihydroxybenzoic acid and 3,5-dihydroxybenzoic acid.

In another aspect, L may represent a C1-4 alkylene group, for instance a C1-3 alkylene group, preferably a C1-2 alkylene group. The alkylene group may be unsubstituted or substituted with one, two or three substituents selected from NH₂, OH, SH, fluorine or COOH, preferably NH₂, OH and fluorine, most preferably NH₂. L may therefore be an ethylene group or a methylene group which is unsubstituted or substituted with one, two or three substituents selected from NH₂, OH, SH, fluorine or COOH, preferably NH₂, OH and fluorine, most preferably NH₂.

Typically, when L represents a C₁₋₂ alkylene group, n is from 1 to 4. Preferably n is from 1 to 3. More preferably n is 1 or 2.

Typically, L represents a C1-2 alkylene group which is unsubstituted or substituted with NH₂. Preferably L represents an ethylene group substituted with NH₂. Thus, the compound of formula (II) may be 4-hydroxyphenylalanine (tyrosine):

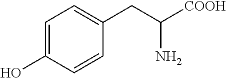 [SMILES: NC(Cc1ccc(O)cc1)C(=O)O]

Preferably, the compound of formula (II) is L-tyrosine:

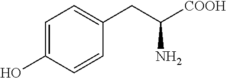 [SMILES: N[C@@H](Cc1ccc(O)cc1)C(=O)O]

Thus, the cocrystal of the invention comprises any substituted 2-oxo-1-pyrrolidinyl triazole compound according to formula (I) and any coformer which is a carboxylic acid of formula (II). The cocrystal of the invention may consist essentially of any 2 substituted 2-oxo-1-pyrrolidinyl triazole compound according to formula (I) and any coformer which is a carboxylic acid of formula (II). The cocrystal of the invention may consist of any substituted 2-oxo-1-pyrrolidinyl triazole compound according to formula (I) and any coformer which is a carboxylic acid of formula (II). Preferably, the substituted 2-oxo-1-pyrrolidinyl triazole of formula (I) is (4R)-1-[(5-chloro-1H-1,2,4-triazol-1-yl)methyl]-4-(3,4,5-trifluorophenyl)pyrrolidin-2-one (formula (Ia)).

The term “consists essentially of” refers to a cocrystal in which the total content of the compound formula (I) and the compound of formula (II) accounts for at least 95% by weight of the cocrystal, typically at least 98% by weight of the cocrystal, preferably at least 99% by weight of the cocrystal.

Thus, the cocrystal may comprise, consist essentially of or consist of (4R)-1-[(5-chloro-1H-1,2,4-triazol-1-yl)methyl]-4-(3,4,5-trifluorophenyl)pyrrolidin-2-one, and a carboxylic acid of formula (II) as described herein. When the cocrystal comprises the 2-oxo-1-pyrrolidinyl triazole of formula (I) and the carboxylic acid of formula (II), the cocrystal may also comprise solvent molecules. Thus, the cocrystal may be a solvate. The cocrystal may therefore comprise, consist essentially of or consist of the 2-oxo-1-pyrrolidinyl triazole of formula (I) and the carboxylic acid of formula (II) and solvent molecules. For instance the cocrystal may be a hydrate. The cocrystal may therefore comprise, consist essentially of or consist of the 2-oxo-1-pyrrolidinyl triazole of formula (I) and the carboxylic acid of formula (II) and water molecules. The term “consists essentially of” in the context of a cocrystal which is a solvate or hydrate refers to a cocrystal in which the total content of the compound formula (I), the compound of formula (II) and the solvent molecules, for instance water molecules, accounts for at least 95% by weight of the cocrystal, typically at least 98% by weight of the cocrystal, preferably at least 99% by weight of the cocrystal.

Typically, the cocrystal comprises, consists essentially of or consists of (4R)-1-[(5-chloro-1H-1,2,4-triazol-1-yl)methyl]-4-(3,4,5-trifluorophenyl)pyrrolidin-2-one and a carboxylic acid of formula (II) in which L is a bond and n is from 1 to 4, preferably from 1 to 3, more preferably 1 or 2. Typically, the cocrystal comprises, consists essentially of or consists of (4R)-1-[(5-chloro-1H-1,2,4-triazol-1-yl)methyl]-4-(3,4,5-trifluorophenyl)pyrrolidin-2-one and a carboxylic acid selected from the group consisting of a hydroxybenzoic acid, a dihydroxybenzoic acid and a trihydroxybenzoic acid. Preferably, the cocrystal comprises, consists essentially of or consists of (4R)-1-[(5-chloro-1H-1,2,4-triazol-1-yl)methyl]-4-(3,4,5-trifluorophenyl)pyrrolidin-2-one and a hydroxybenzoic acid or a dihydroxybenzoic acid.

Thus, typically, the cocrystal comprises, consists essentially of or consists of (4R)-1-[(5-chloro-1H-1,2,4-triazol-1-yl)methyl]-4-(3,4,5-trifluorophenyl)pyrrolidin-2-one and carboxylic acid selected from the group consisting of 2-hydroxybenzoic acid, 3-hydroxybenzoic acid, 4-hydroxybenzoic acid, 2,3-dihydroxybenzoic acid, 2,4-dihydroxybenzoic acid or 3,5-dihydroxybenzoic acid. Preferably, the cocrystal comprises, consists essentially of or consists of (4R)-1-[(5-chloro-1H-1,2,4-triazol-1-yl)methyl]-4-(3,4,5-trifluorophenyl)pyrrolidin-2-one and carboxylic acid selected from the group consisting of 4-hydroxybenzoic acid, 2,4-dihydroxybenzoic acid and 3,5-dihydroxybenzoic acid.

The cocrystal may comprise, consist essentially of or consist of (4R)-1-[(5-chloro-1H-1,2,4-triazol-1-yl)methyl]-4-(3,4,5-trifluorophenyl)pyrrolidin-2-one and 4-hydroxybenzoic acid.

The cocrystal may comprise, consist essentially of or consist of (4R)-1-[(5-chloro-1H-1,2,4-triazol-1-yl)methyl]-4-(3,4,5-trifluorophenyl)pyrrolidin-2-one and 4-hydroxybenzoic acid.

The cocrystal may comprise, consist essentially of or consist of (4R)-1-[(5-chloro-1H-1,2,4-triazol-1-yl)methyl]-4-(3,4,5-trifluorophenyl)pyrrolidin-2-one and 4-hydroxybenzoic acid.

The cocrystal may comprise, consist essentially of or consist of (4R)-1-[(5-chloro-1H-1,2,4-triazol-1-yl)methyl]-4-(3,4,5-trifluorophenyl)pyrrolidin-2-one and a carboxylic acid of formula (11) in which L represents a C1-2 alkylene group which is unsubstituted or substituted with NH₂. The cocrystal comprise, consist essentially of or consist of (4R)-1-[(5-chloro-1H-1,2,4-triazol-1-yl)methyl]-4-(3,4,5-trifluorophenyl)pyrrolidin-2-one and a carboxylic acid of formula (II) in which L represents an ethylene group substituted with NH₂. Thus, the cocrystal may comprise, consist essentially of or consist of (4R)-1-[(5-chloro-1H-1,2,4-triazol-1-yl)methyl]-4-(3,4,5-trifluorophenyl)pyrrolidin-2-one and tyrosine, preferably L-tyrosine.

The cocrystal may comprise, consist essentially of or consist of (4R)-1-[(5-chloro-1H-1,2,4-triazol-1-yl)methyl]-4-(3,4,5-trifluorophenyl)pyrrolidin-2-one and a carboxylic acid selected from the group consisting of 4-hydroxybenzoic acid, 4-hydroxybenzoic acid, 4-hydroxybenzoic acid and tyrosine, preferably L-tyrosine.

Typically, the melting point of the cocrystal is at least 85° C. For instance, the melting point of the cocrystal may be at least 90° C., at least 95° C., at least 100° C. or at least 110° C. A higher melting point is advantageous because higher melting point materials are more easily processed into pharmaceutical products. For instance, materials with higher melting points are more resistant to degradation during particle-size reduction.

Typically, the molar ratio of the substituted 2-oxo-1-pyrrolidinyl triazole of formula (I) to the carboxylic acid of formula (II) is from 3:1 to 1:3. Preferably the molar ratio of the substituted 2-oxo-1-pyrrolidinyl triazole of formula (I) to the carboxylic acid of formula (II) is from 1:1 to 1:2. For instance, the molar ratio of the substituted 2-oxo-1-pyrrolidinyl triazole of formula (I) to the carboxylic acid of formula (II) may be 1:1. The molar ratio of the substituted 2-oxo-1-pyrrolidinyl triazole of formula (I) to the carboxylic acid of formula (II) may be 1:2. A molar ratio of the substituted 2-oxo-1-pyrrolidinyl triazole of formula (I) to the carboxylic acid of formula (II) of around 1:2 is beneficial from a formulation perspective, as the increased quantity of coformer relative to API makes it easier to prepare low dosage compositions of the API at an accurate dosage because for any given weight of cocrystal, a smaller proportion of API is present. On the other hand, a molar ratio of the substituted 2-oxo-1-pyrrolidinyl triazole of formula (I) to the carboxylic acid of formula (II) of around 1:1 is useful for formulation pharmaceutical compositions in which the therapeutic dosage is high, because for any given weight of cocrystal, a greater proportion of API is present.

Thus, the cocrystal of the invention may comprise a substituted 2-oxo-1-pyrrolidinyl triazole of formula (I) and 3,5-dihydroxybenzoic acid in a molar ratio of from 0.8:1 to 1.2:1, for instance in a molar ratio of from 0.9:1 to 1.1:1, preferably in a molar ratio of 1:1.

The cocrystal of the invention may comprise a substituted 2-oxo-1-pyrrolidinyl triazole of formula (I) and 2,4-dihydroxybenzoic acid in a molar ratio of from 1:1.5 to 1:2.5, typically in a molar ratio of from 1:1.8 to 1:2.2, preferably in a molar ratio of 1:2.

Typically, the cocrystals of the invention are stable after storage at elevated temperature and humidity. For instance, the cocrystals of the invention remain unchanged after storage at 40° C. and 75% relative humidity for 1 week. The benefit of cocrystals that are stable in these sorts of conditions is that they do not require any special storage conditions, making storage and transportation more straightforward, regardless of the climate the cocrystals are being used in. Moreover, stability under such storage conditions is a good indication that longer term stability will be achieved under controlled conditions, such as low temperature and low humidity.

### Pharmaceutical Composition

The invention also provides a pharmaceutical composition comprising a cocrystal as described herein and a pharmaceutically acceptable excipient.

Suitable pharmaceutically acceptable excipients are well known to those skilled in the art and include pharmaceutically acceptable carriers (e.g. a saline solution, an isotonic solution), diluents, adjuvants, fillers, buffers, preservatives, anti-oxidants, lubricants, stabilisers, solubilisers, surfactants (e.g. wetting agents), masking agents, colouring agents, flavouring agents and sweetening agents. Suitable carriers, diluents, excipients, etc. can be found in standard pharmaceutical texts. See, for example, Handbook for Pharmaceutical Additives, 2nd Edition (eds. M. Ash and I. Ash), 2001 (Synapse Information Resources, Inc., Endicott, New York, USA), Remington's Pharmaceutical Sciences, 20th edition, pub. Lippincott, Williams & Wilkins, 2000; and Handbook of Pharmaceutical Excipients, 2nd edition, 1994.

The pharmaceutical composition may be suitable for oral, buccal, parenteral, nasal, topical, ophthalmic or rectal administration, or a form suitable for administration by inhalation or insufflation. Typically, the pharmaceutical composition is suitable for oral administration.

For oral administration, the pharmaceutical composition may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulphate). The tablets may be coated by methods well known in the art. Liquid preparations for oral administration may take the form of, for example, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Pharmaceutical compositions for oral administration may be suitably formulated to give controlled release of the active compound.

For buccal administration, the pharmaceutical compositions may take the form of tablets or lozenges formulated in conventional manner.

The pharmaceutical composition may be for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions or emulsions in oily or aqueous vehicles, and may contain formulatory agents such as suspending, stabilising, preserving and/or dispersing agents. Alternatively, the active ingredients may be in powder form for constitution with a suitable vehicle, e.g. sterile pyrogen-free water, before use.

In addition to the formulations described above, the pharmaceutical composition may also be formulated as a depot preparation. Such long-acting formulations may be administered by implantation or by intramuscular injection.

For nasal administration or administration by inhalation, the pharmaceutical composition may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The pharmaceutical compositions may, if desired, be presented in a pack or dispenser device which may contain one or more unit dosage forms containing the active ingredient. The pack or dispensing device may be accompanied by instructions for administration. For topical administration the pharmaceutical composition may be conveniently formulated in a suitable ointment containing the active component suspended in one or more pharmaceutically acceptable carriers. Particular carriers include, for example, mineral oil, liquid petroleum, propylene glycol, polyoxyethylene, polyoxypropylene, emulsifying wax and water. Alternatively, the pharmaceutical composition may be formulated as a suitable lotion containing the compound suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the pharmaceutical composition may be formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration the pharmaceutical composition may be formulated as an ointment such as petrolatum.

For rectal administration the pharmaceutical composition may be conveniently formulated as suppositories. These can be prepared by mixing the compound with a suitable non-irritating excipient which is solid at room temperature but liquid at rectal temperature and so will melt in the rectum to release the active component. Such materials include, for example, cocoa butter, beeswax and polyethylene glycols.

The cocrystal or pharmaceutical composition as described herein may be administered to the subject daily, typically once per day, to deliver a therapeutically effective amount of the compound of formula (I). The cocrystal or pharmaceutical composition may be administered as a single unit dosage. Useful dosages of the compound of formula (I) are described in WO 2015/014785.

The invention also provides a cocrystal as described herein or a pharmaceutical composition as described herein for use in medicine. In particular, the invention provides a cocrystal as described herein or a pharmaceutical composition as described herein for use in the treatment of a cognitive disorder. The cocrystal as described herein or a pharmaceutical composition as described herein may be for use in enhancing or improving cognitive ability or counteracting cognitive decline.

The terms “enhancing or improving cognitive ability” and “counteracting cognitive decline” used throughout this specification shall mean promoting cognitive function (affecting impaired cognitive function in the subject so that it more closely resembles the function of an aged-matched normal, unimpaired subject, including affecting states in which cognitive function is reduced compared to a normal subject) and preserving cognitive function (affecting normal or impaired cognitive function such that it does not decline or does not fall below that observed in the subject upon first presentation or diagnosis, e.g. to the extent of expected decline in the absence of treatment). The suitability of the compounds according to the present invention for conditions associated with enhancement or improvement of cognitive ability may be tested through assays that are well known in the art. Such assays include in particular the novel object recognition tests, as well as the Y-maze test, word recall tests and digit symbol substitution tests (DSST).

The cognitive disorder may be selected from autism, dyslexia, attention deficit hyperactivity disorder, obsessive compulsive disorders, psychosis, bipolar disorders, depression (major depressive disorder), Tourette's syndrome and disorders of learning in children, adolescents and adults, Age Associated Memory Impairment, Age Associated Cognitive Decline, Parkinson's Disease, Down's Syndrome, traumatic brain injury, Huntington's Disease, Progressive Supranuclear Palsy (PSP), HIV infection, stroke, vascular diseases, Pick's or Creutzfeldt-Jacob diseases, multiple sclerosis (MS), other white matter disorders and drug-induced cognitive worsening, Alzheimer's disease, schizophrenia, Lewy-bodies disease, front-temporal lobe degeneration, vascular narrowing or blockage in the brain (i.e. vascular dementia also known as multi-infarct dementia), head trauma, subjective cognitive decline and mild cognitive impairment.

The cocrystal or pharmaceutical composition as described herein are typically for use in treating a cognitive disorder selected from subjective cognitive decline, Age Associated Memory Impairment, mild cognitive impairment, Alzheimer's disease, cognitive impairment in major depressive disorder and cognitive impairment in a subject with remitted depression following multiple episodes of major depressive disorder.

The cocrystal or pharmaceutical composition as described herein may be for use in treating subjective cognitive decline. The cocrystal or pharmaceutical composition as described herein may be for use in treating Age Associated Memory Impairment. The cocrystal or pharmaceutical composition as described herein may be for use in treating mild cognitive impairment. The cocrystal or pharmaceutical composition as described herein may be for use in treating Alzheimer's disease. The cocrystal or pharmaceutical composition as described herein may be for use in treating cognitive impairment in major depressive disorder. The cocrystal or pharmaceutical composition as described herein may be for use in treating cognitive impairment in a subject with remitted depression following multiple episodes of major depressive disorder.

The cocrystal or pharmaceutical composition as described herein may be for use in treating disorders associated with loss of cognition, for example the cocrystal or pharmaceutical composition as described herein may be for use in treating disorders in learning and memory. The c cocrystal or pharmaceutical composition as described herein may be for use in treating disorders in learning and memory, Parkinson's disease, Huntington's disease, Tourette's syndrome, and obsessive-compulsive disorder.

Typically, the cocrystal or pharmaceutical composition as described herein is administered orally.

Typically the subject is a mammal. Preferably the subject is a human.

### Method of Manufacture

Compounds of formula (I) may be prepared as described in WO 2015/014785.

The skilled person would be well aware of techniques for preparing cocrystals. For instance, the cocrystals of the invention may be prepared by liquid-assisted grinding. Thus, a process for producing a cocrystal comprising a substituted 2-oxo-1-pyrrolidinyl triazole compound of formula (I) and a carboxylic acid of formula (II) may comprise mixing the compound of formula (I) with the carboxylic acid of formula (II) in the presence of a solvent. Typically the process for producing a cocrystal comprising a substituted 2-oxo-1-pyrrolidinyl triazole compound of formula (I) and a carboxylic acid of formula (II) comprises grinding the compound of formula (I) with the carboxylic acid of formula (II) in the presence of a solvent. Grinding the compound of formula (I) and a carboxylic acid of formula (II) may comprise ball milling the compound of formula (I) and a carboxylic acid of formula (II) in the presence of the solvent.

Typically the volume of solvent used is low, such that it is not sufficient to dissolve all of the compound of formula (I) and carboxylic acid of formula (II). Typically, the solvent is an organic solvent, for instance a polar organic solvent. For instance, the solvent may be selected from acetonitrile and isopropylalcohol.

The process may further comprise removing the solvent. Typically, the solvent is removed by drying in air at room temperature.

To aid or improve cocrystal formation, the process may comprise further steps following mixing the compound of formula (I) with the carboxylic acid of formula (II) in the presence of a solvent. Such steps may include:
Adding a second solvent to the product of the mixing step described above to form a suspension or solution;
Treating the suspension or solution to one or more heating and cooling cycles;
Removing the second solvent, for instance by drying in air at room temperature;
Adding a third solvent to the product left after the second solvent is removed form a suspension or solution and sonicating the suspension or solution;
Removing the third solvent, for instance by drying in air at room temperature.

The cocrystals of the invention may also be prepared by solvent-based methods. For instance, the process for producing a cocrystal comprising a substituted 2-oxo-1-pyrrolidinyl triazole compound of formula (I) and a carboxylic acid of formula (II) may comprise:
Dissolving the substituted 2-oxo-1-pyrrolidinyl triazole compound of formula (I) in a first solvent to form a first solution;
Dissolving the carboxylic acid of formula (II) in a second solvent to form a second solution;
Mixing the first solution and the second solution at a first temperature;
Cooling the mixture of the first and second solutions to a second temperature.

The first temperature is typically from 10° C. to 100° C., for instance from 25° C. to 75° C., preferably from 40° C. to 60° C. The second temperature is typically from −15° C. to 25° C., for instance from −5° C. to 10° C.

The first solvent is typically an organic solvent. For instance, the first solvent may be selected from the group consisting of isopropyl acetate, toluene and methyl isobutyl ketone. The second solvent is typically an organic solvent. Usually the second solvent is different from the second solvent. For instance, the second solvent may be tetrahydrofuran (THF).

The process may optionally comprise one or more of the following steps:
Adding an antisolvent to the cooled mixture of the first and second solutions;
Adding seeds of the cocrystal of interest to the cooled mixture of the first and second solutions;
Removing the first and second solvents, and the antisolvent if present, typically by drying in air at room temperature.

The antisolvent is a solvent in which the cocrystal product is less soluble compared to the first and second solvents. Typically, the antisolvent is an apolar organic solvent, for instance n-heptane.

The process for producing a cocrystal comprising a substituted 2-oxo-1-pyrrolidinyl triazole compound of formula (I) and a 2,4-dihydroxybenzoic acid may preferably comprise:
dissolving the substituted 2-oxo-1-pyrrolidinyl triazole compound of formula (I) in a first solvent to form a first solution, preferably wherein the first solvent is toluene;
dissolving the 2,4-dihydroxybenzoic acid in a second solvent to form a second solution, preferably wherein the second solvent is THF;
mixing the first solution and the second solution at a first temperature, wherein the first temperature is typically between 20 and 80° C., preferably between 40 and 60° C., typically about 50° C.;
cooling the mixture of the first and second solutions to a second temperature, wherein the second temperature is typically between 0 and 10° C., preferably about 5° C.;
adding a first volume of antisolvent to the cooled mixture of the first and second solutions, preferably wherein the antisolvent is n-heptane;
optionally adding seeds of a cocrystal comprising 2-oxo-1-pyrrolidinyl triazole compound of formula (I) and 2,4-dihydroxybenzoic acid to the cooled mixture of the first and second solutions;
optionally adding a second volume of antisolvent to the mixture;
increasing the temperature of the mixture to a third temperature, preferably wherein the third temperature is room temperature, and stirring the mixture;
optionally removing the solvents to isolate cocrystals of a substituted 2-oxo-1-pyrrolidinyl triazole compound of formula (I) and a 2,4-dihydroxybenzoic acid.

The process for producing a cocrystal comprising a substituted 2-oxo-1-pyrrolidinyl triazole compound of formula (I) and a 3,5-dihydroxybenzoic acid may preferably comprise:
dissolving the substituted 2-oxo-1-pyrrolidinyl triazole compound of formula (I) in a first solvent to form a first solution, preferably wherein the first solvent is methyl isobutyl ketone;
dissolving the 3,5-dihydroxybenzoic acid in a second solvent to form a second solution, preferably wherein the second solvent is THF;
mixing the first solution and the second solution at a first temperature, wherein the first temperature is typically between 20 and 80° C., preferably between 40 and 60° C., typically about 50° C.;
cooling the mixture of the first and second solutions to a second temperature, wherein the second temperature is typically between 0 and 10° C., preferably about 5° C.;
adding a first volume of antisolvent to the cooled mixture of the first and second solutions, preferably wherein the antisolvent is n-heptane;
optionally adding seeds of a cocrystal comprising 2-oxo-1-pyrrolidinyl triazole compound of formula (I) and 3,5-dihydroxybenzoic acid to the cooled mixture of the first and second solutions;
optionally adding a second volume of antisolvent to the mixture;
optionally removing the solvents to isolate cocrystals of a substituted 2-oxo-1-pyrrolidinyl triazole compound of formula (I) and a 3,5-dihydroxybenzoic acid.

### EXAMPLES

Abbreviations

| Acronym | Meaning |
|---|---|
| 2,4-DHBA | 2,4-dihydroxybenzoic acid |
| 3,5-DHBA | 3,5-dihydroxybenzoic acid |
| 4-HBA | 4-hydroxybenzoic acid |
| ¹H NMR | Proton Nuclear Magnetic Resonance |
| ACN | Acetonitrile |
| API | Active Pharmaceutical Ingredient |
| ASR | Analytical Service Report |
| ca. | Approximately |
| CCD | Charge Coupled Detector |
| DCM | Dichloromethane |
| DMSO | Dimethyl sulfoxide |
| DSC | Differential Scanning Calorimetry |
| DVS | Dynamic Vapour Sorption |
| eq | Equivalents |
| EtOAc | Ethyl acetate |
| EtOH | Ethanol |
| FBRM | Focussed Beam Reflectance Measurement |
| FID | Flame Ionisation Detector |
| FTIR | Fourier Transform Infrared |
| GC | Gas Chromatography |
| GVS | Gravimetric Vapour Sorption |
| H₂O | Water |
| HPLC | High Performance Liquid Chromatography |
| HS | Head Space |
| HSM | Hot Stage Microscopy |
| IC | Ion Chromatography |
| ID | Identification |
| IDR | Intrinsic Dissolution Rate |
| IP | Intellectual Property |
| IPA | 2-Propanol |
| IPAC | Isopropyl acetate |
| ISA | Ionic Strength Adjusted |
| MeCN | Acetonitrile |
| MEK | Methyl ethyl ketone |
| MeOH | Methanol |
| MIBK | Methyl isobutyl ketone |
| mp | Melting point |
| MS | Mass Spectroscopy |
| N/A | Not Applicable |
| NMR | Nuclear Magnetic Resonance |
| No. | Number |
| n-PrOH | 1-Propanol |
| PDA | Photodiode Array |
| PLM | Polarised Light Microscopy |
| PTFE | Polytetrafluoroethylene |
| RH | Relative Humidity |
| RT | Room Temperature |
| SCXRD | Single Crystal X-Ray Diffraction |
| SEM | Scanning Electron Microscope |
| TBME | tert-Butyl methyl ether |
| TFA | Trifluoroacetic acid |
| TGA | Thermal Gravimetric Analysis |
| THF | Tetrahydrofuran |
| TIC | Total Ion Current |
| USP | United States Pharmacopeia |
| UV | Ultraviolet |
| VH-XRPD | Variable Humidity X-Ray Powder Diffraction |
| vol | Volumes |
| VT-XRPD | Variable Temperature X-Ray Powder Diffraction |
| XRPD | X-Ray Powder Diffraction |

### Materials and Methods

### Active Pharmaceutical Ingredient (API)

The API used in all of the cocrystal experiments described below was (4R)-1-[(5-chloro-1H-1,2,4-triazol-1-yl)methyl]-4-(3,4,5-trifluorophenyl)pyrrolidin-2-one in its free base form. Throughout the examples, this is referred to as the “API”.

### X-Ray Powder Diffraction (XRPD)

Bruker AXS C2 GADDS: XRPD diffractograms were collected on a Bruker AXS C2 GADDS diffractometer using Cu Kα radiation (40 kV, 40 mA), an automated XYZ stage, a laser video microscope for auto sample positioning and a Våntec-500 2-dimensional area detector. X-ray optics consists of a single Göbel multilayer mirror coupled with a pinhole collimator of 0.3 mm. The beam divergence, i.e. the effective size of the X-ray beam on the sample, was approximately 4 mm. A θ-θ continuous scan mode was employed with a sample-detector distance of 20 cm which gives an effective 2θ range of 1.5°-32.5°. Typically, the sample was exposed to the X-ray beam for 120 seconds. The software used for data collection and analysis was GADDS for Win7/XP and Diffrac Plus EVA respectively.

Ambient conditions: Samples run under ambient conditions were prepared as flat plate specimens using powder as received without grinding. Samples were prepared and analysed on a glass slide, by lightly pressed the powder to obtain a flat surface for analysis.

Non-ambient conditions: For variable temperature (VT-XRPD) experiments samples were mounted on an Anton Paar DHS 900 hot stage at ambient conditions. The sample was then heated to the appropriate temperature at 10° C./min and subsequently held isothermally for 2 minutes before data collection. Samples were prepared and analysed on a silicon wafer mounted to the hot stage using a heat-conducting paste.

Bruker AXS D8 Advance: XRPD diffractograms were collected on a Bruker D8 diffractometer using Cu Kα radiation (40 kV, 40 mA) and a θ-2θ goniometer fitted with a Ge monochromator. The incident beam passes through a 2.0 mm divergence slit followed by a 0.2 mm anti-scatter slit and knife edge. The diffracted beam passes through an 8.0 mm receiving slit with 2.5° Soller slits followed by the Lynxeye Detector. The software used for data collection and analysis was Diffrac Plus XRD Commander and Diffrac Plus EVA respectively. Samples were run under ambient conditions as flat plate specimens using powder as received. The sample was prepared on a polished, zero-background (510) silicon wafer by gently pressing onto the flat surface or packed into a cut cavity. The sample was rotated in its own plane. The details of the data collection method are:
Angular range: 2 to 42° 2θ
Step size: 0.05° 2θ
Collection time: 0.5 s/step (total collection time: 6.40 min)

PANalytical Empyrean: XRPD diffractograms were collected on a PANalytical Empyrean diffractometer using Cu Kα radiation (45 kV, 40 mA) in transmission geometry. A 0.5° slit, 4 mm mask and 0.04 rad Soller slits with a focusing mirror were used on the incident beam. A PIXcel3D detector, placed on the diffracted beam, was fitted with a receiving slit and 0.04 rad Soller slits. The software used for data collection was X'Pert Data Collector using X′Pert Operator Interface. The data were analysed and presented using Diffrac Plus EVA or HighScore Plus. Samples were prepared and analysed in a metal 96 well-plate in transmission mode. X-ray transparent film was used between the metal sheets on the metal well-plate and powders (approximately 1-2 mg) were used as received. The scan mode for the metal plate used the gonio scan axis. The details of the data collection method are:
Angular range: 2.5 to 32.0° 2θ
Step size: 0.0130° 2θ
Collection time: 12.75 s/step (total collection time of 2.07 min)

### Nuclear Magnetic Resonance (NMR)

Solution State NMR: 1H NMR spectra were collected on a Bruker 400 MHz instrument equipped with an auto sampler and controlled by a DRX400 console. Samples were prepared in DMSO-d6 solvent, unless otherwise stated. Automated experiments were acquired using ICON NMR configuration within Topspin software, using standard Bruker-loaded experiments (1H, 13C {1H}, DEPT135). Off-line analysis was performed using ACD Spectrus Processor. For non-routine spectroscopy (2D NMR and variable temperature NMR), data were acquired through the use of Topspin alone.

### Differential Scanning Calorimetry (DSC)

TA Instruments Q2000: DSC data were collected on a TA Instruments Q2000 equipped with a 50 position auto sampler. Typically, 0.5 3 mg of each sample, in a pin-holed aluminium pan, was heated at 10° C./min from 25° C. to 235° C. A purge of dry nitrogen at 50 mlmL/min was maintained over the sample. The instrument control software was Advantage for Q Series and Thermal Advantage and the data were analysed using Universal Analysis or TRIOS.

TA Instruments Discovery DSC: DSC data were also collected on a TA Instruments Discovery DSC equipped with a 50 position auto-sampler. Typically, 0.5 3 mg of each sample, in a pin-holed aluminium pan, was heated at 10° C./min from 25° C. to 235° C. A purge of dry nitrogen at 50 mlmL/min was maintained over the sample. The instrument control software was TRIOS and the data were analysed using TRIOS or Universal Analysis.

Thermo-Gravimetric Analysis (TGA)

TA Instruments Discovery TGA: TGA data were collected on a TA Instruments Discovery TGA, equipped with a 25 position auto-sampler. Typically, 5 10 mg of each sample was loaded onto a pre-tared aluminium DSC pan and heated at 10° C./min from ambient temperature to 350° C. A nitrogen purge at 25 mlmL/min was maintained over the sample. The instrument control software was TRIOS and the data were analysed using Universal Analysis.

### Polarised Light Microscopy (PLM)

Leica LM/DM Polarised Light Microscope: Samples were analysed on a Leica LM/DM polarised light microscope with a digital video camera for image capture. A small amount of each sample was placed on a glass slide, with immersion oil, and covered with a glass slip. The sample was viewed with appropriate magnification and partially polarised light, coupled to a X false-colour filter. Images were captured using StudioCapture or Image ProPlus software.

Nikon LM/DM Polarised Light Microscope: Samples were studied on a Nikon SMZ1500 polarised light microscope with a digital video camera connected to a DS Camera control unit DS-L2 for image capture. The sample was viewed with appropriate magnification and partially polarised light, coupled to a X false colour filter.

### Scanning Electron Microscopy (SEM)

Data were collected on a Phenom Pro Scanning Electron Microscope. A small quantity of sample was mounted onto an aluminium stub using conducting double-sided adhesive tape. A thin layer of gold was applied using a sputter coater (20 mA, 120 s).

### Gravimetric Vapour Sorption (GVS)

Sorption isotherms were obtained using a SMS DVS Intrinsic moisture sorption analyser, controlled by DVS Intrinsic Control software. The sample temperature was maintained at 25° C. by the instrument controls. The humidity was controlled by mixing streams of dry and wet nitrogen, with a total flow rate of 200 ml/min. The relative humidity was measured by a calibrated Rotronic probe (dynamic range of 1.0-100% RH), located near the sample. The weight change, (mass relaxation) of the sample as a function of % RH was constantly monitored by a microbalance (accuracy ±0.005 mg). Typically, 5-30 mg of sample was placed in a tared mesh stainless steel basket under ambient conditions. The sample was loaded and unloaded at 40% RH and 25° C. (typical room conditions). A moisture sorption isotherm was performed as outlined below (2 scans per complete cycle). The standard isotherm was performed at 25° C. at 10% RH intervals over a 0-90% RH range. Typically, a double cycle (4 scans) was carried out. Data analysis was carried out within Microsoft Excel using the DVS Analysis Suite.

TABLE 1
Method for SMS DVS Intrinsic experiments

| Parameter | Value |
|---|---|
| Adsorption - Scan 1 | 40-90 |
| Desorption, Adsorption - Scan 2 | 90-0, 0-40 |
| Intervals (% RH) | 10 |
| Number of Scans | 4 |
| Flow rate (mL/min) | 200 |
| Temperature (° C.) | 25 |
| Stability (° C./min) | 0.2 |
| Sorption Time (hours) | 6 hour time out |
| Number of cycles | 2 |

The sample was recovered after completion of the isotherm and re-analysed by XRPD.

### Chemical Purity Determination by HPLC

Purity analysis was performed on an Agilent Ip 100/Infinity 111260 series system equipped with a diode array detector and using OpenLAB software. The full method details are provided below:

TABLE 2
HPLC method for chemical purity determinations

| Parameter | Value | | |
|---|---|---|---|
| Type of method | Reverse phase with gradient elution | | |
| Sample Preparation | 0.3 mg/mL in ACN:water 1:1 | | |
| Column | Supelco Ascentis Express C18 2.7 μm | | |
| | 100 × 4.6 mm | | |
| Column Temperature (° C.) | 25 | | |
| Injection (μL) | 5 | | |
| Detection: Wavelength, | 211, 90 | | |
| Bandwidth (nm) | | | |
| Flow Rate (mL/min) | 2 | | |
| Phase A | 0.1% TFA in water | | |
| Phase B | 0.085% TFA in acetonitrile | | |
| Timetable | Time (min) | % Phase A | % Phase B |
| | 0 | 95 | 5 |
| | 6 | 5 | 95 |
| | 6.2 | 95 | 5 |
| | 8 | 95 | 5 |

### Ion Chromatography (IC)

Data were collected on a Metrohm 930 Compact IC Flex with 858 Professional autosampler and 800 Dosino dosage unit monitor, using IC MagicNet software. Accurately weighed samples were prepared as stock solutions in a suitable solvent. Quantification was achieved by comparison with standard solutions of known concentration of the ion being analysed. Analyses were performed in duplicate and an average of the values is given unless otherwise stated.

TABLE 3
IC method for cation chromatography

| Parameter | Value |
|---|---|
| Type of method | Cation exchange |
| Column | Metrosep C 4 − 250 (4.0 × 250 mm) |
| Column Temperature (° C.) | Ambient |
| Injection (μL) | Various |
| Detection | Conductivity detector |
| Flow Rate (mL/min) | 0.9 |
| Eluent | 1.7 mM nitric acid |
| | 0.7 mM dipicolinic acid in a 5% acetone |
| | aqueous solution. |

TABLE 4
IC method for anion chromatography

| Parameter | Value |
|---|---|
| Type of method | Anion exchange |
| Column | Metrosep A Supp 5 - 150 (4.0 × 150 mm) |
| Column Temperature (° C.) | Ambient |
| Injection (μL) | Various |
| Detection | Conductivity detector |
| Flow Rate (mL/min) | 0.7 |
| Eluent | 3.2 mM sodium carbonate |
| | 1.0 mM sodium hydrogen carbonate in a 5% |
| | acetone aqueous solution. |

### Sirius Inform

Data were collected on a Sirius inForm instrument fitted with a dual UV Dip Probe attachment and Ag/AgCl combination pH electrode. The electrode was calibrated using the four plus parameters derived from a blank titration. The base titrant was standardised by titration with TRIS. 0.5 M HCl and NaOH aqueous solutions were used as the acid and base titrants respectively for the testing. Stirring was facilitated by a dual overhead stirrer to allow thorough mixing within the vessel, and media was introduced via a capillary bundle attached to a dispensing bank comprised of six precision dispensing units. A Peltier heating jacket was used to maintain the temperature of the titration vessel. Discs were introduced to the vessel via the tablet picker housed in the probe arm, after the desired temperature of the media had been reached. Sirius inForm Assay Design, Control and Refine software were used to design, run and refine data respectively.

### Single Crystal X-Ray Diffraction (SCXRD)

Data were collected on a Rigaku Oxford Diffraction Supernova Dual Source, Cu at Zero, Atlas CCD diffractometer equipped with an Oxford Cryosystems Cobra cooling device. The data were collected using Cu Kα or Mo Kα radiation as stated in the experimental tables. Structures were solved and refined using the Bruker AXS SHELXTL suite or the OLEX2 crystallographic software. Full details can be found in the CIF. Unless otherwise stated, hydrogen atoms attached to carbon were placed geometrically and allowed to refine with a riding isotropic displacement parameter. Hydrogen atoms attached to a heteroatom were located in a difference Fourier synthesis and were allowed to refine freely with an isotropic displacement parameter. A reference diffractogram for the crystal structure was generated using Mercury (Mercury: visualization and analysis of crystal structures. Macrae, Claire F., et al. 2006, J. Appl. Cryst., Vol. 39, pp. 453-457).

### Example 1—Solvent Assessment and Cooling Screen with Citric Acid and 2,4-Dihydroxybenzoic Acid

### General Procedures

### Materials

Commercial chemicals and solvents were purchased from Aldrich or Fluka. The coformers used in this report are shown in Table 5.

TABLE 5
Coformers used in screens

| Set 1 of coformers | | Set 2 of coformers | |
|---|---|---|---|
| Methyl paraben | Valeric acid | L-Glutamic acid | Maleic acid |
| Saccharin | Citric acid | L-(−)-Malic acid | L-(+)-Tartaric acid |
| Nicotinamide | Resorcinol | Adipic acid | Succinic acid |
| Nicotinic acid | Benzoic acid | Levulinic acid | Ascorbic acid |
| Urea | 2,4-Dihydroxy- | Maltol | L-Hydroxyproline |
| | benzoic acid | | |
| 4-Aminobenzoic | LTyrosine | Threonine | Vanilic acid |
| acid | | | |
| 4-Hydroxybenzoic | L-Proline | Salicylic acid | 2,3-Dihydroxybenzoic |
| acid | | | acid |
| Glycine | L-Lactic acid | 3-Hydroxybenzoic | 2,5-Dihydroxybenzoic |
| | | Acid | acid |
| Serine | Propionic acid | 3,5-Dihydroxy- | 3,4-Dihydroxybenzoic |
| | | benzoic acid | acid |

### Dry Grinding and Liquid-Assisted Grinding

Grinding is a traditional method used to reduce particle size or as a way to produce amorphous material. However, the use of liquid assisted grinding (LAG) has proven to be an effective method of forming polymorphs, salts and cocrystals that are unavailable using solvent free methods. The small volume of solvent acts as a catalyst, assisting the ball milling mechanism and greatly increasing the crystallisation kinetics. A mixture of API (18×25 mg) and the coformer (1.0 equiv., see Table 6) were placed in HPLC vials with two 3 mm ball bearings. The materials were either wetted with solvent (10 or 2θ μl μL of n-heptane, isopropanol or acetonitrile). or contained no solvent before being ground for 2 hours at 500 rpm using a Fritsch milling system with an Automaxion adapter. After grinding all solids were initially analysed by XRPD following brief air drying.

### Maturation

For the investigation of crystalline forms, often maturation experiments (or slurry ripening) are performed in various solvents or solvent mixtures and subjected to heat-cool cycles. Repeated heating and cooling cycles may increase the degree of crystallinity or convert a meta-stable state (or out-of-equilibrium state in the case of amorphous material) into a more thermodynamically stable crystalline form. It can also facilitate the formation of salts or cocrystals. The rate and extent of conversion is dependent upon solubility of the input material. Suspensions for maturation were placed in a platform shaker incubator (Heidolph Titramax/Incubator 1000) and subjected to a series of heat-cool cycles from ambient to approximately 50° C. This is achieved by switching the heating on or off every 4 hours. Shaking is maintained throughout. For this study, samples were matured on a shaker mill using heat/cool cycles (8 hr) between RT and 50° C.

### Controlled Evaporation

Crystallisation can be generated by controlled evaporation of a clear, particulate free, solution. This is especially true when the solvent has a relatively high vapour pressure. At approximately constant temperature, the solvent is being removed from the system, thereby increasing the solute concentration. The crystal nucleation and growth is obtained when some maximum supersaturation is reached. This technique also has the advantage that since the samples are slowly evaporated it is often possible to generate large single crystals suitable for SCXRD. Solutions were evaporated at ambient conditions by covering the vials in plastic film or tinfoil with pin holes. The samples were allowed to slowly evaporate to dryness or until a solid appeared at ambient conditions.

### Cooling Crystallisation

Crystallisation can be obtained by lowering the temperature of a clear solution. The solubility of most materials decreases with decreasing temperature, so cooling can be used to generate supersaturation. In many cases however, the solubility of a material remains high even at low temperatures or the solubility changes very little over the temperature range of interest. In these cases, other methods for creation of supersaturation must be considered (such as solvent evaporation). For this study, solutions were cooled from 50° C. to 5° C. at 10° C./min, unless otherwise stated, in a Polar Bear and stirred at this temperature overnight. All solids were filtered and initially analysed by XRPD. Any remaining solutions were normally left to evaporate at ambient conditions.

### Precipitation/Crystallisation by Anti-Solvent Addition

Anti-solvent crystallisation (or drown out crystallisation) is a method commonly used to precipitate material from a solution. The addition of a miscible anti-solvent into a solute solution, reduces the original solubility of the solute, increasing the supersaturation and thus, causing its precipitation. The selected anti-solvent should be miscible with the solvent at any proportion, and the solute should be relatively insoluble in it. Solutions were treated with equivalent anti-solvent volumes. Observations were made before a second volume equivalent was added. Once a precipitate had formed the solids were filtered using positive pressure and analysed using XRPD.

### Sonication

Gums were sonicated with n-heptane or in the absence of solvent.

### Characterisation of Supplied Material

The received API material was characterised using a wide range of techniques to investigate the solid form and chemical properties of this material. A summary of the results is shown in Table 6.

TABLE 6
Characterisation data for API
API

| XRPD | | Crystalline - matches calculated XRPD from single |
|---|---|---|
| | | crystal structure - Form A |
| HPLC Purity | | 99.5% |
| ¹H-NMR | | Taken as reference |
| | | 10 proton environments identified |
| TGA | | Decomposition from above 200° C. |
| DSC | | Sharp endotherm, onset temperature: 85.3° C. |
| | | (78 J/g) |
| | | Exotherm, onset temperature: 202.8° C. |
| | | (22 J/g) |
| GVS | | 0.05% w/w reversible water uptake between |
| | | 0-90% RH |
| | | Unchanged by XRPD post-GVS |
| Scanning IC | | No anions or cations observed |
| Storage | 25° C./ | Purity - 7 days: 99.5%, 14 days: 99.5% |
| | 97% RH | XRPD - Unchanged |
| | 40° C./ | Purity - 7 days: 99.5%, 14 days: 99.5% XRPD - |
| | 75% RH | Unchanged |

The XRPD data confirmed that the supplied API sample is crystalline and is consistent with the simulated XRPD pattern from the single crystal X-ray structure for Form A. A sharp endothermic event was observed in the DSC thermogram at 85.3° C., corresponding to the melt. An exothermic event at 202.8° C. is observed prior to decomposition. The TGA thermogram confirmed that the sample is anhydrous with no weight loss prior to sample decomposition above 200° C. The supplied material is 99.5% pure as determined by HPLC. It is stable after 14 days at 25° C./75% RH and 40° C./97% RH and is not hygroscopic (maximum uptake of 0.05% w/w by GVS with no form change observed).

### Example 1—Solvent Assessment and Cooling Screen with Citric Acid and 2,4-Dihydroxybenzoic Acid

### Procedure 1

API (25 mg) was dispensed to a PLC vial with a magnetic stirrer. At room temperature an aliquot of solvent was added (5 vol.=125 μL) and stirred at 25° C. for at least 5 minutes. A visual assessment was made. Solutions were held at 25° C. and suspensions were placed at 50° C. with stirring for at least 5 minutes. A visual assessment was made and if a suspension was observed, further aliquots of solvent were added at 25° C. and the above steps repeated until a maximum of 50 volumes were added (Table 7).

TABLE 7
Observations from solubility assessment

| | | 5 vol | | 10 vol | | 50 vol | |
|---|---|---|---|---|---|---|---|
| Sample ID | Solvent | 25° C. | 50° C. | 25° C. | 50° C. | 25° C. | 50° C. |
| ARP-1725-06-01 | n-heptane | X | X | — | — | — | X |
| ARP-1725-06-02 | Ethyl acetate | ✓ | — | — | — | — | — |
| ARP-1725-06-03 | 2-Propanol | X | ✓ | — | — | — | — |
| ARP-1725-06-04 | MEK | ✓ | — | — | — | — | — |
| ARP-1725-06-05 | Acetone | ✓ | — | — | — | — | — |
| ARP-1725-06-06 | Ethanol | X | ✓ | — | — | — | — |
| ARP-1725-06-07 | Tetrahydrofuran | ✓ | — | — | — | — | — |
| ARP-1725-06-08 | Acetonitrile | ✓ | — | — | — | — | — |
| ARP-1725-06-09 | Nitromethane | ✓ | — | — | — | — | — |
| ARP-1725-06-10 | Acetone:water (95:5 v/v) | X | ✓ | — | — | — | — |
| ARP-1725-42-01 | Diethyl ether | X | — | X | — | ✓ | — |
| ARP-1725-42-02 | IPAC | ✓ | — | — | — | — | — |
| ARP-1725-42-03 | MIBK | ✓ | — | — | — | — | — |
| ARP-1725-42-04 | Water | X | X | X | X | X | X |
| ARP-1725-42-05 | TBME | X | X | X | ✓ | — | — |
| ARP-1725-42-06 | 2-methyl-1-propanol | X | ✓ | — | — | — | — |
| ARP-1725-42-07 | 1,4-Dioxane | ✓ | — | — | — | — | — |
| ARP-1725-42-08 | Toluene | X | ✓ | — | — | — | — |
| ARP-1725-42-09 | Ethanol:Water (9:1 v/v) | ✓ | — | — | — | — | — |
| ARP-1725-42-10 | IPA:Water (9:1 v/v) | X | ✓ | — | — | — | — |

X = suspension evident,
✓ = clear solution

Following the solubility assessment, 1.1 mol eq of citric acid (83 μL, 1 M in THF) was added to the solutions (at 25 or 50° C.) or suspensions (at 50° C.) with the sample ID: ARP-1725-06-XX, XX=01-10. A 1.1 mol eq of 2,4-dihydroxybenzoic acid (2,4-DITBA) (83 μL, 1 M in THF) was added to the solutions with sample TD: ARP-1725-42-XX=01-10. For the citric acid screen the solutions at 50° C. had a cooling ramp applied from 50° C. to 25° C. at a rate of 0.25° C./min and then from 25° C. to 5° C. at 0.1° C. For the solutions at 25° C., a cooling ramp from 25° C. to 5° C. at 0.1° C./min was applied. For the 2,4-DHBA screen, the solutions at 50° C. were cooled to 5° C. at 0.1° C./min and the solutions at 25° C. were also cooled to 5° C. at 0.1° C./min. Observations were made immediately after the acid addition and at 5° C. (Table 8).

TABLE 8
Observations after acid additions and at 5° C.

| Sample ID | Solvent | Acid | After acid addition | Observation at 5° C. |
|---|---|---|---|---|
| ARP-1725-06- | n-Heptane | Citric | Some solids on vial | Oily/gummy residue on |
| 01 | | acid | walls, some possible | vial walls clear solution |
| | | | dissolution | |
| ARP-1725-06- | Ethyl acetate | | Clear solution | Clear solution |
| 02 | | | | |
| ARP-1725-06- | 2-Propanol | | Clear solution | Clear solution |
| 03 | | | | |
| ARP-1725-06- | MEK | | Clear solution | Clear solution |
| 04 | | | | |
| ARP-1725-06- | Acetone | | Clear solution | Clear solution |
| 05 | | | | |
| ARP-1725-06- | Ethanol | | Clear solution | Clear solution |
| 06 | | | | |
| ARP-1725-06- | Tetrahydrofuran | | Clear solution | Clear solution |
| 07 | | | | |
| ARP-1725-06- | Acetonitrile | | Clear solution | Clear solution |
| 08 | | | | |
| ARP-1725-06- | Nitromethane | | Clear solution | Clear solution |
| 09 | | | | |
| ARP-1725-06- | Acetone:water | | Clear solution | Clear solution |
| 10 | (95:5 v/v) | | | |
| ARP-1725-42- | Diethyl ether | 2,4- | Clear solution | White gum on vial walls |
| 01 | | Dihydroxybenzoic | | |
| ARP-1725-42- | IPAC | acid | Clear solution | Clear solution |
| 02 | | | | |
| ARP-1725-42- | MIBK | | Clear solution | Clear solution |
| 03 | | | | |
| ARP-1725-42- | Water | | Suspension | Thick white solid |
| 04 | | | | suspension |
| ARP-1725-42- | TBME | | Clear solution | Clear solution |
| 05 | | | | |
| ARP-1725-42- | 2-Methyl-1- | | Clear solution | Clear solution |
| 06 | propanol | | | |
| ARP-1725-42- | 1,4-Dioxane | | Clear solution | Clear solution |
| 07 | | | | |
| ARP-1725-42- | Toluene | | Clear solution | Clear solution |
| 08 | | | | |
| ARP-1725-42- | Ethanol:Water | | Clear solution | Clear solution |
| 09 | (9:1 v/v) | | | |
| ARP-1725-42- | IPA:Water (9:1 | | Clear solution | Clear solution |
| 10 | v/v) | | | |

Leaving the citric acid screening samples to further evaporate over several days led to gums being formed. The gums were sonicated with 250 μL n-heptane for 30 minutes and further observations made. Once again all samples remained as gums and were evaporated and dried in a vacuum oven. Once being removed from the vacuum oven the samples were still gums and no further work was done on the samples.

An anti-solvent addition was applied to the 2,4-DHBA screening samples. Volumes of n-heptane were added to a maximum of a 1:5 solvent:anti-solvent ratio. For the water and diethyl ether solutions a specific aliquot of n-heptane was added (Table). Solids produced were analysed using XRPD. Samples that were solutions were evaporated.

TABLE 9
Observations on anti-solvent addition

| | | | Observation after | |
|---|---|---|---|---|
| | | | max volume of | Observation |
| | | Volumes of | anti-solvent | after |
| Sample ID | Solvent | anti-solvent | addition at 5° C. | evaporation |
| ARP-1725-42-01 | Diethyl | 750 μL | White ppt | — |
| | ether | | | |
| ARP-1725-42-02 | IPAC | 5 vol. equiv. | White ppt and gum | — |
| ARP-1725-42-03 | MIBK | 5 vol. equiv. | White gum | — |
| ARP-1725-42-04 | Water | 500 μL | White ppt | — |
| ARP-1725-42-05 | TBME | 2 vol. equiv. | White ppt | — |
| ARP-1725-42-06 | 2-Methyl-1- | 5 vol. equiv. | White ppt | — |
| | propanol | | | |
| ARP-1725-42-07 | 1,4-Dioxane | 5 vol. equiv. | Turbid soln. no | White gum |
| | | | obvious ppt | |
| ARP-1725-42-08 | Toluene | 5 vol. equiv. | White ppt | — |
| ARP-1725-42-09 | Ethanol/H₂O | 5 vol. equiv. | Clear solution | White Gum |
| | (9:1 v/v) | | | |
| ARP-1725-42-10 | IPA/H₂O | 5 vol. equiv. | Turbid soln. no | White gum |
| | (9:1 v/v) | | obvious ppt | |

### Procedure 2

API (25 mg) was weighed into an HPLC vial and 50 vol. (1.25 mL) of water added. The sample was placed at 50° C. for 5 min and 1.1 eq of 2,4-DHBA (1M in THF) was added. The sample (ARP-1725-50-01) was then cooled from 50 to 5° C. at 0.1° C./min. Stirring at 400 rpm was maintained throughout. The resulting gum was sonicated for 30 min producing a fine precipitate that was analysed by XRPD (ARP-1725-50-01_before anti-solvent). The sample was then treated with n-heptane (600 μL), before being re-analysed by XRPD (ARP-1725-50-01_after anti-solvent).

### Results and Discussion

API showed good solubility in most of the organic solvents tested. Water, diethyl ether and n-heptane are the only exceptions and thus were identified as potential anti-solvents for the various cocrystal screens. The citric-acid screen did not produce any solid material that could be analysed by XRPD.

The 2,4-DHBA screen resulted in the 2,4-DHBA Pattern 1 cocrystal being formed after anti-solvent addition when using the solvents: toluene, IPAC and MIBK (Table 10). The formation of the cocrystal also took place in water, however there was still a significant amount of the free base API also present. A further investigation into the formation of the cocrystal in water was conducted to see whether or not the n-heptane anti-solvent addition was required for the formation of the cocrystal. A mixture of the cocrystal and API was present before the anti-solvent addition as well as after (Table 11). Owing to the fact a mixture was obtained This procedure was not investigated further.

The results of the solubility assessment were used to select anti-solvents and solvents for the cocrystal screens discussed in Section 8. Furthermore, the results of the 2.4 dihydroxybenzoic acidDHBA cocrystal formation experiments were used to assist in further investigation to obtain the 2,4-DHBA Pattern 1 cocrystal (Example 3).

TABLE 10
Results from the 2,4-DHBA screen

| Sample ID | Solvent | Procedure | XRPD result |
|---|---|---|---|
| ARP-1725-42-01 | Diethyl ether | Anti-solvent | API only |
| ARP-1725-42-02 | IPAC | Anti-solvent | 2,4-DHBA-Pattern 1 |
| | | | (poor pattern) |
| ARP-1725-42-03 | MIBK | Anti-solvent | 2,4-DHBA-Pattern 1 |
| | | | (poor pattern) |
| ARP-1725-42-04 | Water | Anti-solvent | Mixture API and 2,4- |
| | | | DHBA-Pattern 1 |
| ARP-1725-42-05 | TBME | Anti-solvent | API only (poor |
| | | | pattern) |
| ARP-1725-42-06 | 2-Methyl-1- | Anti-solvent | API only |
| | propanol | | |
| ARP-1725-42-07 | 1,4-Dioxane | Anti-solvent | — |
| | | followed by | |
| | | evaporation | |
| ARP-1725-42-08 | Toluene | 5 vol equiv. | 2,4-DHBA-Pattern 1 |
| ARP-1725-42-09 | Ethanol/H₂O | Anti-solvent | — |
| | (9:1 v/v) | followed by | |
| | | evaporation. | |
| ARP-1725-42-10 | IPA/H₂O | Anti-solvent | — |
| | (9:1 v/v) | followed by | |
| | | evaporation | |

Samples were gums so not analysed

TABLE 11
Results of the further investigations into formation of the 2,4-DHBA cocrystal in water

| Sample ID | Solvent | Observations | XRPD |
|---|---|---|---|
| ARP-1725-50-01_before | Water | After cooling a gum was | Mixture API and |
| anti-solvent | | obtained which when sonicated | 2,4-DHBA |
| | | gave a fine precipitate | Pattern 1 |
| ARP-1725-50-01_after | Water and n- | White precipitate | Mixture API and |
| anti-solvent | heptane | | 2,4-DHBA |
| | | | Pattern 1 |

### Example 2 Cocrystal Screen

### Cocrystal Screen Procedures

Four cocrystal screens were performed with 36 coformers and a range of procedures to optimise the possibility of finding a cocrystal of API. Cocrystal screens 1 and 2 were carried out with the first set of coformers and screens 3 and 4 with the second set. Based on the additional procedures used, suffixes were added to the initial sample ID as outlined (Sample ID_Y, Y=B—F).

### Cocrystal Screen 1

The sample IDs for this screen are ARP-1725-08-XX_Y (XX=01-12, Y=B—F) and ARP-1725-16-XX_Y (XX=01-06, Y=B—F).

API (25 mg) and 1 molar equivalent of coformer were weighed into a vial and were ground for 2 hours in the presence of 20 μL of n-heptane (Procedure 1a, ID=XX). Post grinding, all the samples that were solids were characterised by XRPD analysis.

The samples that were not crystalline or did not display a novel XRPD Pattern were then ground for 2 hours with 10 μL of a new solvent, isopropanol (Procedure 1b, ID=XX_B) and all solids characterised by XRPD.

Physical mixtures or gums were then treated with up to 10 volumes of TBME. If the material did not dissolve the samples were subjected to maturation cycles (Procedure 1d-m, ID=XX_C) for 4 days. If a clear solution resulted, the samples were left to evaporate at ambient conditions. (Procedure id-c, ID=XX_D). After evaporation, vials that contained solid white powders or single crystals were evaluated using XRPD. The samples from maturation that contained very fine suspensions were filtered under positive pressure and the solids analysed using XRPD. The samples that became clear during the maturation cycles were left open to evaporate before being assessed.

For the gums observed from evaporation, 250 μL of n-heptane was added and the sample placed in the sonication bath for 30 minutes (Procedure 1e, ID=XX_E). No solids were formed so all remaining samples were left to evaporate at ambient conditions before drying under vacuum (Procedure if, ID=XX_F).

### Cocrystal Screen 2

The sample IDs for this screen are ARP-1725-11-XX_Y (X=01-12, Y=B F) and ARP-1725-16-XX (X=07-12, Y=B—F).

Equimolar amounts of API (25 mg) and the coformer were weighed into a vial and were ground for 2 hours in the absence of any solvent (Procedure 2a, ID=XX). Post grinding, all the samples were characterised by XRPD analysis.

If physical mixtures or gums were obtained 10 μL of acetonitrile (Procedure 2b, ID=XX_B) was added to these and the samples ground for a further 2 hours. All solid samples were analysed using XRPD.

All samples that were liquids and physical mixtures were then treated with 5 or 10 volumes of IPAC. If the material did not dissolve in a maximum of 10 volumes the samples were subjected to maturation cycles (Procedure 2d-m, ID=XX_C) for 4 days. If a clear solution resulted, the samples were left to evaporate at ambient conditions. (Procedure 2d-c, ID=XX_D). After evaporation, vials that contained solid white powders or single crystals were evaluated using XRPD.

The samples from maturation that contained very fine suspensions were filtered under positive pressure and the solids analysed using XRPD. The samples that became clear during the maturation cycles were left open to evaporate before being assessed. For the gums observed, following solvent evaporation and RT/50° C. maturation cycles, 250 pL of n-heptane was added and the sample placed in the sonication bath for 30 minutes (Procedure 2e, ID=XX_E). The solids were filtered under positive pressure and analysed by XRPD. If gums were persistent the solvent was left to evaporate at ambient conditions before drying under vacuum (Procedure 2f, ID_F).

### Cocrystal Screen 3

The sample IDs for this screen are ARP-1725-38-XX_Y (X=01-18, Y=B E).

API (25 mg) and 1 molar equivalent of coformer were weighed into a vial and were ground for 2 hours (Section 5.2.1) in the presence of 10 μL of acetonitrile. Post grinding, the samples were uncapped and left to evaporate before the solids were analysed by XRPD analysis.

The steel grinding balls were removed from any samples that were not novel crystalline cocrystals and 20 volumes of n-heptane was added before the samples were placed in the sonication bath for 30 minutes (Procedure 3b; ID=XX_B). The samples that contained white solids were analysed by XRPD.

Any physical mixtures and gums were treated with 200 pL of iso-propanol and then subjected to maturation cycles (RT/50° C.) (Procedure 3c, ID=XX_C) for four days. Those that produced solids were analysed by XRPD while the vials containing clear solutions were subjected to an anti-solvent addition (Procedure 3e, ID=XX_E).

### Cocrystal Screen 4

Stock solutions of API (500 mg) in toluene (5 vols, 2.5 mlmL) and TBME (10 vols, 5 mlmL) as well as 1 M stock solutions of the co-formers in THE or water were prepared. For both screens L-glutamic acid was added as a solid. In some instances, the API was weighed directly into the vial (25 mg) and the required volume of solvent added.

The stock solutions of API were dispensed into HPLC vials (ca. 25 mg per vial) and 1.1 equivalent of coformer was then added as solid or stock solution and the sample stirred at ambient temperatures. The solutions were left to equilibrate at 50° C. for ca. an hour before they were cooled from 50° C. to 5° C. at 0.1° C./min (Procedure 4a, ID=XX), and all solids analysed by XRPD. If a sample produced a solid after the coformer addition, it was also heated and cooled before being analysed by XRPD.

The samples in toluene and TBME were then treated as outlined below and the sample IDs for the Toluene and TBME screen are as follows:

Toluene screen: ARP-1725-54-XX_Y, XX=01-18, Y=B, C

TBME screen: ARP-1725-57-XX_Y, XX=01-18, Y=B, C

### Toluene Screen:

Antisolvent addition was performed for any clear solutions using diethyl ether (Procedure 4c, ID=XX_C). However, after 5 total volume equivalents had been added at 5° C. all the solutions remained clear. The diethyl ether was evaporated off using a gentle air flow, directed across the top of the vials. Two samples produced solids that were analysed by XRPD (sample IDs unchanged) while the remaining 10 samples were reconstituted with 4 volumes of toluene. 7 of the 10 samples remained as white precipitates and thus were analysed by XRPD (sample IDs unchanged). Anti-solvent, n-heptane, was added to the three clear solutions had (Procedure 4c, ID=XX_C). The resulting precipitates were analysed by XRPD. The turbid solutions or solids from the initial Procedure 4a were subjected to maturation cycles for 3 days (Procedure 4b, ID=XX_B). Following maturation any clear solutions were subjected to anti-solvent addition (Procedure 4c, ID=XX_C). As diethyl ether did not prove to be an ideal anti-solvent with the first attempt, n-heptane was used. All solid samples were analysed using XRPD. Only one gum remained at the end of this screen that was not analysed.

### TBME Screen:

Following procedure 4a, the samples that remained as clear solutions underwent anti-solvent additions with n-heptane (Procedure 4c, ID=XX_C). The remaining vials showing turbid solutions or white suspensions were placed in the maturation chamber for 3 days (Procedure 4b, ID=XX_B). All but one sample remained as a clear solution after 3 days in the maturation chamber. The sample was analysed by XRPD and the rest were subjected to anti-solvent addition (n-heptane, Procedure 4c, ID=XX_C). Only one gum remained at the end of the screen.

### Cocrystal Screen Results and Discussion

Four different cocrystal screens were carried out using a total of 36 different coformers. Different sets of solvents and crystallisation conditions were used throughout the screens. Any solid material that resulted from these screens was analysed by XRPD. New phases were named after the coformer (e.g, 2,4-DHBA Pattern 1 for a new phase from an experiment with 2,4 dihydroxybenzoic acid—acronyms given in summary of hits (Table 27)). The results from Cocrystal Screens 1-4 are shown in Table 12 to Table 26. In each instance the initial sample ID is presented in the left hand column. Depending on the further treatment of the samples these are amended with a suffix as outlined in the relevant column (i.e. for Procedure 1b in Cocrystal Screen 1 the sample ID is ARP-1725-08-01_B, Table 13). These are grouped by coformer in order to compare the results with different coformers across the screens.

Cocrystals were formed with three of the 36 coformers investigated. From Cocrystal Screens 1 and 2 it was possible to prepare two new materials with 4 hydroxybenzoic acid (4-HBA) and 2,4-dihydroxybenzoic acid (2,4-DHBA). The latter was also obtained during further solubility assessments when 2,4-DHBA was added to the samples (Example 1). After expanding the search and investigating a further 18 coformers with hydroxy and benzoic acid functional groups, one additional cocrystal was prepared with 3,5-dihydroxybenzoic acid. All three of the cocrystals were initially found using the liquid assisted grinding method. Sample ARP-1725-11-07 (4-HBA cocrystal) was also prepared through evaporative methods during the screening phase (Table 14).

The three potential cocrystals were characterised using DSC, 1H NMR, TGA and by evaluating their stability with XRPD after 7 or 8 days of being stored at 40° C./75% RH. All three cocrystals were unchanged by XRPD after storage at 40° C./75% RH and none had significant amounts of solvent present (Table 28). The 4 HBA cocrystal appeared to have a similar melt to the starting material (ca. 85° C.). However, the 3,5-DHBA cocrystal appeared to have a notably higher melt (108.9° C.). Furthermore, although the 2,4-DHBA cocrystal had an initial endotherm at 63.8° C., a second endotherm was observed at a much higher temperature, 123.6° C., indicating this cocrystal may have a significantly higher melting point than the free form. All initially appeared to have a 1:1 stoichiometry.

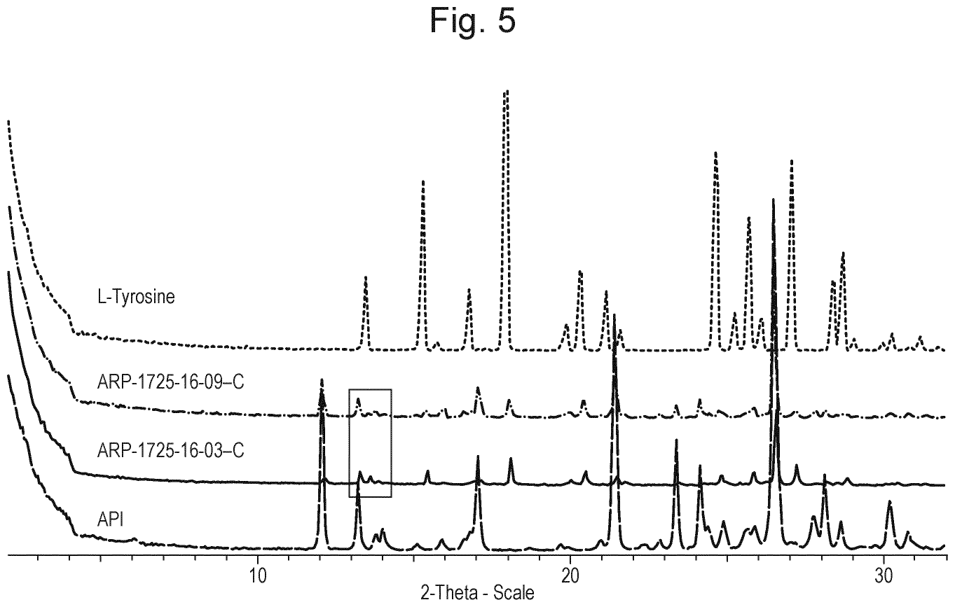

New reflections in the XRPD spectra, compared XRPD spectra for the coformer and API alone, were also observed for L-tyrosine (ARP-1725-16-09_C and ARP-1725-16-03_C). These spectra are shown in FIG. 5. For instance, the region highlighted in the black rectangle exhibits new reflections in the XRPD spectra not observed for API or coformer.

To characterise these forms further, for IP and to assess their potential for development, all three were selected for scale up (Example 3). Initial experiments focused on identifying solution based methods for obtaining the cocrystals that are more easily applicable at larger scales than cocrystal formation via grinding. All three were then obtained at a larger scale and fully characterised.

TABLE 12
Cocrystal Screen 1 results from grinding (Procedure 1a and 1b)

| | | Procedure 1a | Procedure 1b |
|---|---|---|---|
| | | LAG (n-heptane) | LAG (isopropanol): |
| Sample ID | Coformer | (Sample ID) | (Sample ID_B) |
| ARP-1725-08-01 | Methyl paraben | PM | PM |
| ARP-1725-08-02 | Saccharin | PM | PM |
| ARP-1725-08-03 | Nicotinamide | PM | PM |
| ARP-1725-08-04 | Nicotinic Acid | PM | PM |
| ARP-1725-08-05 | Urea | PM | PM |
| ARP-1725-08-06 | 4-Aminobenzoic acid | PM | PM |
| ARP-1725-08-07 | 4-Hydroxybenzoic acid | PM | Reduction in crystallinity |
| ARP-1725-08-08 | Glycine | PM | PM |
| ARP-1725-08-09 | Serine | PM | PM |
| ARP-1725-08-10 | Valeric Acid | API (weak diffraction) | API |
| ARP-1725-08-11 | Citric Acid | PM | API |
| *ARP-1725-08-12 | Resorcinol | PM | PM |
| ARP-1725-16-01 | Benzoic acid | PM | PM |
| ARP-1725-16-02 | 2,4-Dihydroxybenzoic acid | PM and (possible new peaks 18- | Reduction in crystallinity |
| | | 19° 2Θ) | |
| ARP-1725-16-03 | L-Tyrosine | PM | PM |
| ARP-1725-16-04 | L-Proline | PM | PM (new peak at 20.3° 2Θ) |
| ARP-1725-16-05 | L-Lactic acid | API | API |
| ARP-1725-16-06 | Propionic acid | API | API |

PM = physical mixture
*ARP-1725-08-12 was a tacky gum. An aliquot was removed and analysed (ARP-1725-08-12A), the remaining material was left to dry out overnight and analysed again (ARP-1725-08-12B). Both were PM's.

TABLE 13
Cocrystal Screen 1 results from maturation and evaporation (Procedure 1d-m and 1d-c)

| | | Volumes | | |
|---|---|---|---|---|
| Sample ID | Coformer | of TBME | Treatment | Observation/XRPD Result |
| ARP-1725-08-01_C | Methyl paraben | 10 | Maturation | Clear Gum |
| ARP-1725-08-02_C | Saccharin | 10 | Maturation | PM |
| *ARP-1725-08-03_C | Nicotinamide | 10 | Maturation | PM |
| ARP-1725-08-04_C | Nicotinic Acid | 10 | Maturation | PM |
| ARP-1725-08-05_C | Urea | 10 | Maturation | Urea (poor diffraction) |
| ARP-1725-08-06_D | 4-Aminobenzoic acid | 5 | Evaporation | PM |
| ARP-1725-08-07_(C and | 4-Hydroxybenzoic acid | 10 | Maturation followed by | 4-HBA Pattern 1 matches |
| D) | | | Evaporation | ARP-1725-11-07_B |
| ARP-1725-08-08_C | Glycine | 10 | Maturation | PM |
| ARP-1725-08-09_C | Serine | 10 | Maturation | PM |
| ARP-1725-08-10_D | Valeric Acid | 10 | Evaporation | API (with preferred orientation) |
| ARP-1725-08-11_D | Citric Acid | 5 | Evaporation | Clear Gum |
| ARP-1725-08-12_D | Resorcinol | 5 | Evaporation | Clear Gum |
| ARP-1725-16-01_C | Benzoic acid | 10 | Maturation | Benzoic acid |
| ARP-1725-16-02_C | 2,4-dihydroxybenzoic acid | 10 | Maturation | API (reduction in crystallinity) |
| ARP-1725-16-03_C | L-Tyrosine | 10 | Maturation | Tyrosine Pattern 1 |
| ARP-1725-16-04_C | L-Proline | 10 | Maturation | PM |
| ARP-1725-16-05_D | L-Lactic acid | 10 | Evaporation | Clear Gum |
| ARP-1725-16-06_C | Propionic acid | 10 | Maturation | API |

PM = physical mixture
*Crystalline material was retrieved from the mother liquor of sample ARP-1725-08-03_C: XRPD Result: PM, ARP-1725-08-03_CML

TABLE 14
Cocrystal Screen 1 results from sonication, evaporation
and vacuum drying (Procedure 1e and 1f)

| | | Observation after Procedure 1e, | Observation after Procedure 1f, |
|---|---|---|---|
| | | sonication, | evaporation and vacuum dry/ |
| Sample ID | Coformer | Sample ID_E | XRPD result, Sample ID_F |
| ARP-1725-08-01 | Methyl paraben | White gum | White solid - PM |
| ARP-1725-08-11 | Citric acid | Clear gum | Clear Gum |
| ARP-1725-08-12 | Resorcinol | White gum | Clear Gum |
| ARP-1725-16-05 | L-Lactic acid | Clear gum | Clear Gum |

PM = physical mixture

TABLE 15
Cocrystal Screen 2 results from grinding (Procedure 2a and 2b)

| | | Procedure 2a Dry Grinding | Procedure 2b LAG (Acetonitrile) |
|---|---|---|---|
| Sample ID | Coformer | Sample ID | Sample ID_B |
| ARP-1725-11-01 | Methyl paraben | PM | PM |
| ARP-1725-11-02 | Saccharin | PM | PM |
| ARP-1725-11-03 | Nicotinamide | PM | PM |
| ARP-1725-11-04 | Nicotinic Acid | PM | PM |
| ARP-1725-11-05 | Urea | API: reduction crystallinity | PM |
| ARP-1725-11-06 | 4-Aminobenzoic acid | PM | PM |
| ARP-1725-11-07 | 4-Hydroxybenzoic acid | PM (new weak reflections | 4-HBA Pattern 1 |
| | | 8-9° 2Θ) | |
| ARP-1725-11-08 | Glycine | PM | PM (reduction in crystallinity) |
| ARP-1725-11-09 | Serine | PM | PM (reduction in crystallinity) |
| ARP-1725-11-10 | Valeric Acid | API | Clear liquid |
| ARP-1725-11-11 | Citric Acid | PM | Citric acid |
| ARP-1725-11-12 | Resorcinol | PM | Clear liquid |
| ARP-1725-16-07 | Benzoic acid | PM | PM |
| ARP-1725-16-08 | 2,4-Dihydroxybenzoic acid | PM | 2,4-DHBA Pattern 1 |
| ARP-1725-16-09 | L-Tyrosine | PM | PM |
| ARP-1725-16-10 | L-Proline | PM | PM |
| ARP-1725-16-11 | L-Lactic acid | API | Clear liquid |
| ARP-1725-16-12 | Propionic acid | API | Clear liquid |

PM = physical mixture

TABLE 16
Cocrystal Screen 2 results from maturation and evaporation (Procedure 2d-m and 2d-c)

| | | Volumes | | Observation/XRPD |
|---|---|---|---|---|
| Sample ID | Coformer | of IPAC | Treatment | Result |
| ARP-1725-11-01_C | Methyl paraben | 5 | Evaporation | Gum |
| ARP-1725-11-02_(C and | Saccharin | 10 | Maturation then | PM |
| D) | | | evaporation | |
| *ARP-1725-11-03_C | Nicotinamide | 10 | Maturation | Nicotinamide (poor |
| | | | | diffraction) |
| ARP-1725-11-04_C | Nicotinic Acid | 10 | Maturation | Nicotinic acid |
| ARP-1725-11-05_C | Urea | 10 | Maturation | Urea (poor diffraction) |
| ARP-1725-11-06_D | 4-Aminobenzoic acid | 5 | Evaporation | PM |
| ARP-1725-11-07_B | 4-Hydroxybenzoic acid | N/A | N/A | N/A |
| ARP-1725-11-08_C | Glycine | 10 | Maturation | PM |
| ARP-1725-11-09_C | Serine | 10 | Maturation | PM |
| ARP-1725-11-10_D | Valeric Acid | 5 | Evaporation | API (with preferred |
| | | | | orientation) |
| ARP-1725-11-11_C | Citric Acid | 10 | Maturation | Gum |
| ARP-1725-11-12_D | Resorcinol | 5 | Evaporation | Gum |
| ARP-1725-16-07_D | Benzoic acid | 5 | Evaporation | White Gum - sonication |
| ARP-1725-16-08_B | 2,4-Dihydroxybenzoic acid | N/A | N/A | N/A |
| ARP-1725-16-09_C | L-Tyrosine | 10 | Maturation | Tyrosine Pattern 1 |
| ARP-1725-16-10_C | L-Proline | 10 | Maturation | L-Proline (trace API) |
| ARP-1725-16-11_D | L-Lactic acid | 5 | Evaporation | Clear Gum |
| ARP-1725-16-12_D | Propionic acid | 5 | Evaporation | API (with preferred |
| | | | | orientation) |

PM = physical mixture
*Crystalline material was retrieved from the mother liquor of sample ARP-1725-11-03_C: XRPD Result: PM, ARP-1725-11-03_CML

TABLE 17
Cocrystal Screen 2 results after sonication, evaporation and vacuum drying (Procedure 2e and 2f)

| | | Observation after Prodcedure 2e, | Observation after Procedure 2f, |
|---|---|---|---|
| | | sonication/XRPD results. | evaporation and vacuum dry/XRPD |
| Sample ID | Coformer | Sample ID_E | result. Sample ID_F |
| ARP-1725-11-01 | Methyl paraben | White gum | White solid/PM |
| ARP-1725-11-12 | Resorcinol | Clear gum | Clear Gum |
| ARP-1725-16-07 | Benzoic acid | White powder/API | N/A |
| ARP-1725-16-11 | L-Lactic acid | Clear gum | Clear Gum |

TABLE 18
Cocrystal Screen 3 results from grinding in MeCN (Procedure 3a)

| | | Observations | |
|---|---|---|---|
| Sample ID | Coformer | (before XRPD) | XRPD |
| ARP-1725-38-01 | L-Glutamic acid | White powder | PM |
| ARP-1725-38-02 | L-(−)-Malic acid | Clear liquid | — |
| ARP-1725-38-03 | Adipic acid | White powder | PM |
| ARP-1725-38-04 | Levulinic acid | Clear liquid | — |
| ARP-1725-38-05 | Maltol | Wet paste | PM/small differences |
| ARP-1725-38-06 | Threonine | White powder | PM (poor XRPD pattern) |
| ARP-1725-38-07 | Salicylic acid | Wet paste | Mainly Salicylic acid |
| ARP-1725-38-08 | 3-Hydroxybenzoic acid | Wet paste | 3-hydroxybenzoic acid only |
| ARP-1725-38-09 | 3,5-Dihydroxybenzoic acid | Wet paste | 3,5-DHBA Pattern 1 |
| ARP-1725-38-10 | 3,4-Dihydroxybenzoic acid | Wet paste | No Bragg peaks |
| ARP-1725-38-11 | 2,5-Dihydroxybenzoic acid | Wet paste | Poor pattern (peaks match coformer) |
| ARP-1725-38-12 | 2,3-Dihydroxybenzoic acid | Wet paste | Poor pattern (peaks match coformer) |
| ARP-1725-38-13 | Vanillic acid | Brown powder | PM |
| ARP-1725-38-14 | L-Hydroxyproline | White powder | PM |
| ARP-1725-38-15 | Ascorbic Acid | White powder | PM |
| ARP-1725-38-16 | Succinic acid | Wet paste | PM |
| ARP-1725-38-17 | L-(+)-Tartaric acid | Liquid and crystals | Poor pattern (API only) |
| ARP-1725-38-18 | Maleic acid | Wet paste | PM, poor pattern |

PM = physical mixture

TABLE 19
Cocrystal Screen 3 results from sonication in n-heptane (Procedure 3b)

| | | Observations after | |
|---|---|---|---|
| Sample ID | Coformer | sonication | XRPD Result |
| ARP-1725-38-01_B | L-Glutamic acid | White solid ppt | PM |
| ARP-1725-38-02_B | L-(−)-Malic acid | Clear gum | — |
| ARP-1725-38-03_B | Adipic acid | White solid ppt | PM |
| ARP-1725-38-04_B | Levulinic acid | Clear gum | — |
| ARP-1725-38-05_B | Maltol | White solid ppt | Small differences |
| | | | but DSC similar to API |
| ARP-1725-38-06_B | Threonine | White solid ppt | PM |
| ARP-1725-38-07_B | Salicylic acid | White gum | — |
| ARP-1725-38-08_B | 3-Hydroxybenzoic acid | White gum | — |
| ARP-1725-38-10_B | 3,4-Dihydroxybenzoic acid | White gum | — |
| ARP-1725-38-11_B | 2,5-Dihydroxybenzoic acid | White gum | — |
| ARP-1725-38-12_B | 2,3-Dihydroxybenzoic acid | White gum | — |
| ARP-1725-38-13_B | Vanillic acid | Brown solid ppt | PM |
| ARP-1725-38-14_B | L-Hydroxyproline | White solid ppt | PM |
| ARP-1725-38-15_B | Ascorbic Acid | White solid ppt | PM |
| ARP-1725-38-16_B | Succinic acid | White solid ppt | PM |
| ARP-1725-38-17_B | L-(+)-Tartaric acid | Clear gum | — |
| ARP-1725-38-18_B | Maleic acid | White gum | — |

PM = Physical mixture,
ppt—precipitate

TABLE 20
Cocrystal Screen 3 results from maturation in IPA and
anti-solvent addition, n-heptane, (Procedure 3c and 3e)

| | | Observations | Observations after | |
|---|---|---|---|---|
| Sample ID | Coformer | after maturation | anti-solvent addition | XRPD Result |
| ARP-1725-38-01_C | L-Glutamic acid | White ppt | N/A | PM |
| ARP-1725-38-02_E | L-(−)-Malic acid | Clear solution | White suspension | API |
| ARP-1725-38-03_E | Adipic acid | Clear solution | White suspension | PM |
| ARP-1725-38-04_E | Levulinic acid | Clear solution | White suspension | API |
| ARP-1725-38-05_E | Maltol | Clear solution | White suspension | API |
| ARP-1725-38-06_C | Threonine | White ppt | N/A | PM |
| ARP-1725-38-07_E | Salicylic acid | Clear solution | White suspension | API |
| ARP-1725-38-08_E | 3-Hydroxybenzoic acid | Clear solution | White suspension | API |
| ARP-1725-38-10_E | 3,4-Dihydroxybenzoic acid | Clear solution | White suspension | API |
| ARP-1725-38-11_E | 2,5-Dihydroxybenzoic acid | Clear solution | White suspension | API |
| ARP-1725-38-12_E | 2,3-Dihydroxybenzoic acid | Clear solution | White suspension | API |
| ARP-1725-38-13_E | Vanillic acid | Clear solution | Brown suspension | PM |
| ARP-1725-38-14_C | L-Hydroxyproline | White ppt | N/A | PM |
| ARP-1725-38-15_C | Ascorbic Acid | White ppt | N/A | PM |
| ARP-1725-38-16_E | Succinic acid | Clear solution | White suspension | PM |
| ARP-1725-38-17_E | L-(+)-Tartaric acid | Clear solution | Gum on bottom of vial | Not performed |
| ARP-1725-38-18_E | Maleic acid | Clear solution | Clear solution | Not performed |

PM = Physical mixture

TABLE 21
Cocrystal Screen 4 (Toluene) results from slow cooling (Procedure 4a)

| | | Observations after | Observations | |
|---|---|---|---|---|
| Sample ID | Coformer | addition of coformer | at 5° C. | XRPD Result |
| ARP-1725-54-01 | L-Glutamic acid | White ppt (GLU ACID) | White ppt + crystals | PM |
| ARP-1725-54-02 | L-(−)-Malic acid | Clear solution | Clear solution | — |
| ARP-1725-54-03 | Adipic acid | White ppt | Fine ppt on vial walls | Not enough material |
| ARP-1725-54-04 | Levulinic acid | Clear solution | Clear solution | — |
| ARP-1725-54-05 | Maltol | White ppt | White ppt on vial walls | PM (poor pattern, limited |
| | | | | material) |
| ARP-1725-54-06 | Threonine | Clear solution | White ppt on vial walls | API |
| ARP-1725-54-07 | Salicylic acid | Clear solution | Clear solution | — |
| ARP-1725-54-08 | 3-Hydroxybenzoic acid | Clear solution | Clear solution + fine | Flat XRPD - insufficient |
| | | | crystalline material on | material |
| | | | vial wall | |
| ARP-1725-54-09 | 3,5-dihydroxybenzoic acid | Clear solution | Clear solution | — |
| ARP-1725-54-10 | 3,4-Dihydroxybenzoic acid | Clear solution | Clear solution | — |
| ARP-1725-54-11 | 2,5-Dihydroxybenzoic acid | Clear solution | Clear solution | — |
| ARP-1725-54-12 | 2,3-Dihydroxybenzoic acid | Clear solution | Clear solution | — |
| ARP-1725-54-13 | Vanillic acid | Clear yellow solution | Clear yellow solution | — |
| ARP-1725-54-14 | L-Hydroxyproline | White ppt | Clear solution | — |
| ARP-1725-54-15 | Ascorbic Acid | Clear solution | White ppt on vial walls | API |
| ARP-1725-54-16 | Succinic acid | White ppt | White ppt + crystals | PM (poor pattern, limited |
| | | | | material) |
| ARP-1725-54-17 | L-(+)-Tartaric acid | Clear solution | Clear solution | — |
| ARP-1725-54-18 | Maleic acid | Clear solution | Clear solution | — |

PM = Physical mixture,
ppt = precipitate

TABLE 22
Cocrystal Screen 4 (Toluene) results from anti-solvent addition (diethyl ether) and then evaporation

| | | After evaporation | Observation after | |
|---|---|---|---|---|
| Sample ID | Coformer | of diethyl ether | Toluene addn | XRPD Result |
| ARP-1725-54-02 | L-(−)-Malic acid | Clear gum | Clear solution | — |
| ARP-1725-54-04 | Levulinic acid | Clear gum | Clear solution | — |
| ARP-1725-54-07 | Salicylic acid | Clear gum | Clear solution | — |
| ARP-1725-54-08 | 3-Hydroxybenzoic acid | Clear gum | Precipitate | Poor pattern/no diffraction |
| ARP-1725-54-09 | 3,5-Dihydroxybenzoic acid | Clear gum | Precipitate | Poor diffraction |
| ARP-1725-54-10 | 3,4-Dihydroxybenzoic acid | Clear gum | Precipitate | 3,4-dihydroxybenzoic acid |
| | | | | only |
| ARP-1725-54-11 | 2,5-Dihydroxybenzoic acid | Clear gum | Precipitate | 2,5-dihydroxybenzoic acid |
| | | | | only |
| ARP-1725-54-12 | 2,3-Dihydroxybenzoic acid | Clear gum | Precipitate | 2,3-dihydroxybenzoic acid |
| ARP-1725-54-13 | Vanillic acid | Precipitate on walls-XRPD | — | PM |
| ARP-1725-54-14 | L-Hydroxyproline | Precipitate on walls-XRPD | — | PM |
| ARP-1725-54-17 | L-(+)-Tartaric acid | Clear gum | Precipitate | Tartaric acid only (PO) |
| ARP-1725-54-18 | Maleic acid | Clear gum | Precipitate | Maleic acid only |

PM = physical mixture

TABLE 23
Cocrystal Screen 4 (Toluene) results
after maturation (Procedure 4b)

| | | Observation | XRPD |
|---|---|---|---|
| Sample ID | Coformer | after maturation | Result |
| ARP-1725-54-01_B | L-Glutamic acid | White ppt | PM |
| ARP-1725-54-03_B | Adipic acid | Clear solution | — |
| ARP-1725-54-05_B | Maltol | Clear solution | — |
| ARP-1725-54-06_B | Threonine | Clear solution | — |
| ARP-1725-54-15_B | Ascorbic Acid | Clear solution | — |
| ARP-1725-54-16_B | Succinic acid | Crystalline | PM |
| | | looking ppt | |

PM = physical mixture

TABLE 24
Cocrystal Screen 4 (Toluene) results after anti-solvent addition (Procedure 4c)

| | | Anti-solvent addn. | | |
|---|---|---|---|---|
| Sample ID | Coformer | Volume equiv. added | Observation | XRPD Result |
| ARP-1725-54-02_C | L-(−)-Malic | 2 vol equiv | White gum + | API poor pattern |
| | acid | | clear solution | |
| ARP-1725-54-03_C | Adipic acid | 1 vol equiv | White ppt | Co-former + some |
| | | | | additional peaks |
| ARP-1725-54-04_C | Levulinic acid | 2 vol equiv | White gum and | API |
| | | | white ppt | |
| ARP-1725-54-05_C | Maltol | 1 vol equiv | White ppt | Similar to coformer |
| | | | | but small differences |
| ARP-1725-54-06_C | Threonine | 1 vol equiv | ppt | API |
| ARP-1725-54-07_C | Salicylic acid | 2 vol equiv | White gum and | PM |
| | | | white ppt | |
| ARP-1725-54-15_C | Ascorbic Acid | 2 vol equiv | Yellow Gum | — |
| ARP-1725-54-16_C | Succinic acid | 2 vol equiv | ppt | PM |

PM = physical mixture

TABLE 25
Cocrystal Screen 4 (TBME) results from slow cooling (Procedure 4a)

| | | Observations after | Observations | XRPD Result/further |
|---|---|---|---|---|
| Sample ID | Coformer | addition of coformer | at 5° C. | treatment |
| ARP-1725-57-01 | L-Glutamic acid | White ppt (GLU ACID) | White ppt | Poor pattern but PM |
| | | | | (Maturation) |
| ARP-1725-57-02 | L-(−)-Malic acid | Clear solution | Clear solution | Antisolvent addition |
| ARP-1725-57-03 | Adipic acid | Clear solution | Clear solution | Anti-solvent addition |
| ARP-1725-57-04 | Levulinic acid | Clear solution | Clear solution | Anti-solvent addition |
| ARP-1725-57-05 | Maltol | Clear solution | Turbid | Maturation |
| ARP-1725-57-06 | Threonine | Clear solution | White ppt | API (maturation) |
| ARP-1725-57-07 | Salicylic acid | Clear solution | Clear solution | Anti-solvent addition |
| ARP-1725-57-08 | 3-Hydroxybenzoic acid | Clear solution | Clear solution | Anti-solvent addition |
| ARP-1725-57-09 | 3,5-Dihydroxybenzoic acid | Clear solution | Clear solution | Anti-solvent addition |
| ARP-1725-57-10 | 3,4-Dihydroxybenzoic acid | Clear solution | Clear solution | Anti-solvent addition |
| ARP-1725-57-11 | 2,5-Dihydroxybenzoic acid | Clear solution | Clear solution | Anti-solvent addition |
| ARP-1725-57-12 | 2,3-Dihydroxybenzoic acid | Clear solution | Clear solution | Anti-solvent addition |
| ARP-1725-57-13 | Vanillic acid | Clear yellow solution | Clear yellow solution | Anti-solvent addition |
| ARP-1725-57-14 | L-Hydroxyproline | Clear solution | ppt on neck of vial | Maturation |
| ARP-1725-57-15 | Ascorbic Acid | Clear solution | Slightly turbid | Maturation |
| ARP-1725-57-16 | Succinic acid | Clear solution | Turbid | Maturation |
| ARP-1725-57-17 | L-(+)-Tartaric acid | Clear solution | Clear solution | Anti-solvent addition |
| ARP-1725-57-18 | Maleic acid | Clear solution | Clear solution | Anti-solvent addition |

ppt = precipitate

TABLE 26
Cocrystal Screen 4 (TBME) results from maturation and anti-solvent addition (Procedure 4b and 4c)

| | | Observations after | Observations after | |
|---|---|---|---|---|
| Sample ID | Coformer | maturation | anti-solvent addition | XRPD Result |
| ARP-1725-57-01_B | L-Glutamic acid | White ppt | N/A | PM |
| ARP-1725-57-02_C | L-(−)-Malic acid | N/A | White solid on vial walls | PM |
| ARP-1725-57-03_C | Adipic acid | N/A | White solid on vial walls | PM |
| ARP-1725-57-04_C | Levulinic acid | N/A | White solid on vial walls | API only |
| ARP-1725-57-05_C | Maltol | Clear solution | White precipitate | PM |
| ARP-1725-57-06_C | Threonine | Clear solution | White precipitate | API only |
| ARP-1725-57-07_C | Salicylic acid | N/A | White solid on vial walls | PM |
| ARP-1725-57-08_C | 3-Hydroxybenzoic acid | N/A | White solid on vial walls | API only |
| ARP-1725-57-09_C | 3,5-Dihydroxybenzoic acid | N/A | Gum and small amount of | Insufficient |
| | | | white solid | material |
| ARP-1725-57-10_C | 3,4-Dihydroxybenzoic acid | N/A | Gum | Not performed |
| ARP-1725-57-11_C | 2,5-Dihydroxybenzoic acid | N/A | White solid on vial walls | API only |
| ARP-1725-57-12_C | 2,3-Dihydroxybenzoic acid | N/A | White solid on vial walls | API only |
| ARP-1725-57-13_C | Vanillic acid | N/A | White suspension | PM |
| ARP-1725-57-14_C | L-Hydroxyproline | Clear solution | White precipitate | PM (mainly API) |
| ARP-1725-57-15_C | Ascorbic Acid | Clear solution | Orange and white ppt | API |
| ARP-1725-57-16_C | Succinic acid | Clear solution | White precipitate | PM (PO) |
| ARP-1725-57-17_C | L-(+)-Tartaric acid | N/A | White solid on bottom | PM (PO) |
| ARP-1725-57-18_C | Maleic acid | N/A | White solid on vial walls | API only |

PM = Physical Mixture

TABLE 27
Summary of characterisation data for the three hits obtained from Cocrystal Screen 1-4.

| Sample ID | ARP-1725-11-07_B | ARP-1725-16-08_B | ARP-1725-38-09 |
|---|---|---|---|
| Coformer | 4-hydroxybenzoic acid | 2,4-dihydroxybenzoic acid | 3,5-dihydroxybenzoic acid |
| | (4-HBA) | (2,4-DHBA) | (3,5-DHBA) |
| Method | Liquid-assisted | Liquid-assisted | Liquid-assisted |
| preparation | grinding (IPA) | grinding (MeCN) | grinding (MeCN) |
| XRPD | 4-HBA Pattern 1 | 2,4-DHBA Pattern 1 | 3,5-DHBA Pattern 1 |
| 1H-NMR | Consistent with structure | Consistent with structure | Consistent with structure |
| | ~1.1 mol. eq 4-HBA | ~1.1 mol. eq 2,4-DHBA | ~1.1 mol. eq 3,5-DHBA |
| TGA | 31% w/w loss from ca. | 33.6% w/w loss from | 0.4% w/w loss from RT-113° C. |
| | 150-254° C. | 130-220° C. | 33.9% w/w loss from 113-245° C. |
| | Followed by continual | Followed by continual | Followed by continual |
| | decomposition | decomposition | decomposition |
| DSC | Endotherm, onset | Endotherm, onset | Endotherm, onset |
| | temperature: 87.5° C. | temperature: 63.8° C. | temperature: 108.9° C. |
| | (64 J/g) Exotherm, | (16 J/g) Endotherm/ | (58 J/g) |
| | onset temperature: | Exotherm, onset | Followed immediately |
| | 116.5° C. (18 J/g) | temperature: 123.6° C. | by a broad exotherm |
| | | (16 J/g) | |
| | | Followed by exothermic event | |
| VT-XRPD | — | Only very slight changes in | — |
| | | XRPD to 110° C. | |
| | | Sample melts somewhere | |
| | | between 110 and | |
| | | 150° C. (Sample ARP-1725-42-08) | |
| 40° C./75% RH | 7 days: Unchanged | 8 days: Unchanged | 7 days: Unchanged |

### Example 3—Scale Up and Characterisation

### Procedure

API (40 mg) was dissolved in 5 vols (200 pL) of solvent (10 volumes (400 pL for TBME) at 50° C. with stirring at 400 rpm. To the solution, 1.1 mol. eq of coformer (67 pL, 2 M stock in THF) was added and all remained clear solutions. A cooling ramp from 50° C. to 5° C. at 0.25° C./minute was applied. All samples were solutions at 5° C. and anti-solvent (n-heptane) was added as 1:1, 1:3 and 1:5 total anti-solvent equivalents. There was at least 5 minutes stirring between later additions. All samples were then stirred at 5° C. overnight.

### Result and Discussion

The 4-HBA Pattern 1 cocrystal was only obtained as a mixture with API (Form A) in IPAC in this investigation (Table 28). For this reason, further solution based methods were attempted to form this cocrystal. Similarly, the 2,4-DHBA Pattern 1 cocrystal was only obtained as mixtures with the free form (Form A, Table 28). However, as the cocrystal had been obtained previously from toluene via solution based methods (Example 1) this solvent was selected for the scale up process. The 3,5-DHBA Pattern 1 cocrystal was successfully obtained from IPAC and MIBK (Table 28). The crystallinities of the two batches appeared comparable and the latter was chosen for the scale up experiments.

TABLE 28
Results of solution based investigations

| | | | Final solvent: anti-solvent | Observation, 5° C., | |
|---|---|---|---|---|---|
| Sample ID | Solvent | Coformer | ratio used and observation | overnight stirring | XRPD |
| ARP-1725-58-01 | IPAC | 4-HBA | 1:5 light gumming in | White suspension | Mixture API and 4-HBA |
| | | | clear solution | | |
| ARP-1725-58-02 | MIBK | | 1:3 white suspension | White suspension | API only |
| ARP-1725-58-03 | Toluene | | 1:5 light gumming in | Clear solution, gum | n/a |
| | | | clear solution | on vial walls | |
| ARP-1725-58-04 | TBME | | 1:3 white suspension | White suspension | API only |
| ARP-1725-58-05 | MeCN | | 1:5 clear solution | Clear solution | n/a |
| ARP-1725-58-06 | IPAC | 2,4-DHBA | 1:5 gumming in clear | White suspension | Mixture API and 2,4-DHBA |
| | | | solution | | Pattern 1 |
| ARP-1725-58-07 | MIBK | | 1:5 light gumming in | White suspension | Mixture API and 2,4-DHBA |
| | | | clear solution | | Pattern 1 |
| ARP-1725-58-08 | Toluene | | 1:5 gumming in clear | White suspension | Mixture API, 2,4-DHBA and |
| | | | solution | | 2,4-DHBA Pattern 1 |
| ARP-1725-58-09 | TBME | | 1:1 white suspension | White suspension | API |
| ARP-1725-58-10 | MeCN | | 1:5 Clear solution | Clear solution | n/a |
| ARP-1725-58-11 | IPAC | 3,5-DHBA | 1:5 heavy gumming in | White suspension | 3,5-DHBA Pattern 1 |
| | | | clear solution | | |
| ARP-1725-58-12 | MIBK | | 1:5 heavy gumming in | White suspension | 3,5-DHBA Pattern 1 |
| | | | clear solution | | |
| ARP-1725-58-13 | Toluene | | 1:5 heavy gumming in | Clear solution, heavy | n/a |
| | | | clear solution | gumming on vial walls | |
| ARP-1725-58-14 | TBME | | 1:5 heavy gumming in | Clear solution, heavy | n/a |
| | | | clear solution | gumming on vial walls | |
| ARP-1725-58-15 | MeCN | | Clear solution | Clear solution | n/a |

### Hydroxybenzoic Acid (4-HBA Pattern 1)

### Procedure

### Preparation of Seeds

API (100 mg) was dispensed into 2 HPLC vials and 1 eq of 4-HBA was added with 2 stainless steel grinding balls to each. Two different quantities (10 and 2θ pL) of IPA was also added to the two vials. The samples were then ground on a planetary mill at 500 rpm for 2 hours and analysed by XRPD.

### Cocrystal Formation

API (100 mg) was dispensed into 5 HPLC vials to which a minimum volume of IPAC, MIBK, Toluene, TBME and IPA were added at 50° C. while stirring at 400 rpm. To these solutions 1.1 mole equivalent of the 4-HBA stock solution (4M in THF) was added. All solutions remained clear upon addition of the coformer and were stirred at 400 rpm for another hour at 50° C. A cooling ramp from 50° C. to 25° C. at 0.25° C./min was applied. After holding at 25° C. for 15 minutes a second cooling ramp from 25° C. to 5° C. at 0.1° C./min was applied. The solids obtained from TBME and IPA were then analysed by XRPD (Table 29). Seeds of 4-HBA were then added to the 5 vials at 5° C. (the seeds dissolved within minutes) and a maturation temperature cycle was applied (4 hours at 5° C., ramp to 25° C. and hold for 4 hours) with stirring for two days. The solids from TBME and IPA samples were re-analysed after 2 days and were physical mixtures or showed the active ingredient only. The solutions were treated with anti-solvent (n-heptane) and the resulting solids filtered and analysed by XPRD and then dried in a vacuum oven at 25° C. for 2 hours, Table 30).

### Results and Discussion

XRPD analysis of the samples from grinding confirmed that 4-HBA Pattern 1 had been obtained in both instances. However, some API Form A may also be present with potentially slightly more in the sample from 10 μL of IPA (ARP 1725-79-01). For this reason, the sample produced from 2θ μL of IPA (ARP 1725-79-02) was then used for seeding in the 4-HBA cocrystal attempts. These two quick grinding experiments show that the 4-HBA cocrystal can be obtained reproducibly by grinding although some optimisation may be required to get a pure phase.

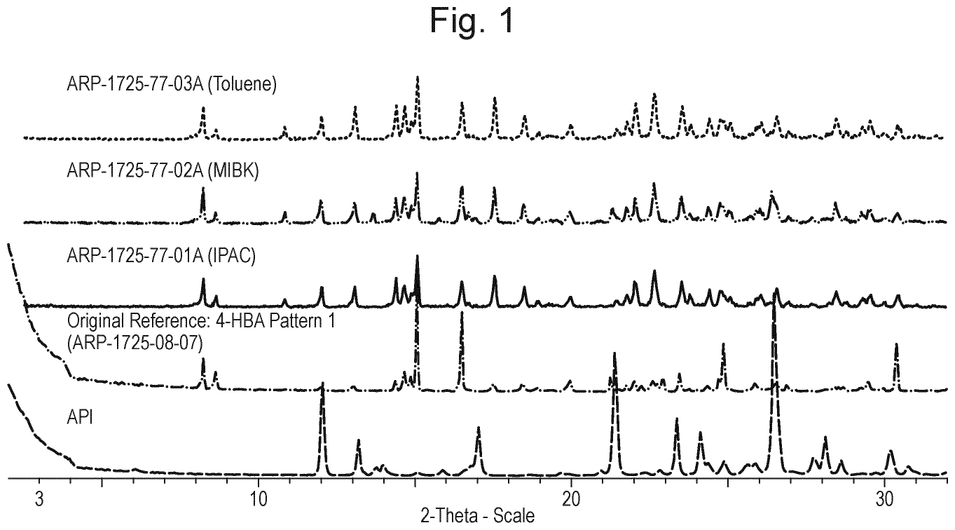

The 4-HBA cocrystal was successfully obtained from IPAC, MIBK and Toluene following antisolvent addition (FIG. 1). The sample (ARP-1725-77-O1B) obtained from IPAC was used to fully characterise the 4 HBA cocrystal and the results are summarised in Table 31.

TABLE 29
Details and observations for the preparation of the 4-HBA cocrystal

| | | Final solvent volumes | | Observation after | |
|---|---|---|---|---|---|
| Sample ID | Solvent | added to API at 50° C. | | cooling to 5° C. | XRPD result |
| ARP-1725-77-01 | IPAc | 2 | vol | Clear solution | — |
| ARP-1725-77-02 | MIBK | 2 | vol | Clear solution | — |
| ARP-1725-77-03 | Toluene | 2 | vol | Clear solution | — |
| ARP-1725-77-04 | TBME | 10 | vol | Precipitate | Physical mixture |
| ARP-1725-77-05 | IPA | 4 | vol | Precipitate | API only (poor pattern) |

TABLE 30
Further details and results following the anti-solvent
addition for the preparation of the 4-HBA cocrystal

| | | Final | | |
|---|---|---|---|---|
| | | solvent: anti- | | |
| Sample ID | Solvent | solvent ratio used | Observation | XRPD result |
| ARP-1725-77-01 | IPAc | 2 vol | White suspension | 4-HBA Pattern 1 |
| ARP-1725-77-02 | MIBK | 2 vol | White suspension | 4-HBA Pattern 1 + small |
| | | | | quantity of API |
| ARP-1725-77-03 | Toluene | 3 vol | White suspension | 4-HBA Pattern 1 |

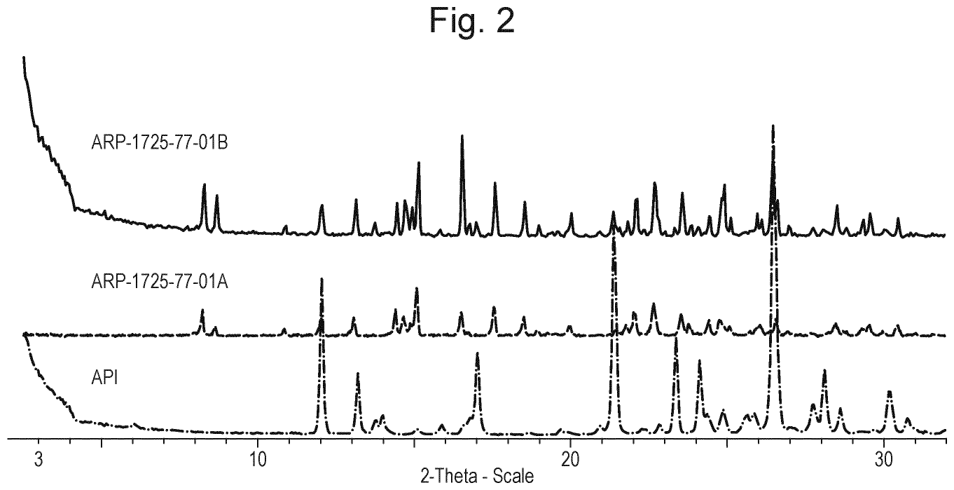

Examination of the 4-HBA cocrystal pre and post isolation showed slight differences (FIG. 2). This may indicate the presence some free form or another crystalline phase.

In the DSC there is a melt endotherm at 87.7° C. and an additional endothermic peak below this melt. VT XRPD on the sample shows some changes in the diffractogram between 60 to 80° C. A visual change was noted at this stage most likely corresponding with the initial endotherm observed by DSC. Melting of the sample was then observed at ca. 86° C. DSCs for the additional two samples obtained from MIBK and Toluene also showed similar thermal behaviour. The 1H NMR data also indicates a slight excess of API. The 4-HBA cocrystal is non-hygroscopic and stable at increased temperatures (40° C.) and at high humidity. There is a small initial mass loss which may indicate the presence of a small amount of residual solvent.

TABLE 31
Summary of characterisation results for cocrystal with 4-hydroxybenzoic acid
4-HBA Pattern 1 (ARP-1725-77-01B)

| Form | | 4-hydroxybenzoic acid (4-HBA) cocrystal |
|---|---|---|
| Method preparation | | Slow cooling in IPAC, MIBK and toluene followed |
| | | by n-heptane anti-solvent additions. |
| XRPD | | 4-HBA Pattern 1 |
| HPLC purity | | 99.8% |
| ¹H-NMR | | Consistent with structure |
| | | 0.85. mol eq 4-HBA |
| TGA | | 29.0% w/w loss from 114-268° C. |
| | | (calc. 1 mol. 4-HBA 29.5%) |
| | | Followed by continual decomposition |
| DSC | | 1^{st} Endotherm, onset temp: 70.6° C. (8 J/g) |
| | | 2^{nd} Endotherm, onset temp: 87.7° C. (56 J/g) |
| | | Broad exotherm, onset temp: 110° C. (19 J/g) |
| VT-XRPD | | XRPD pattern changes slightly on heating between 60° C. |
| | | and 80° C. The sample also changes visually from a white |
| | | powder to an opaquer/translucent material between 60° C. |
| | | and 80° C. |
| GVS | | ~0.11% w/w reversible water uptake between 0-90% |
| | | RH, unchanged by XRPD post-GVS |
| | | Non-hygroscopic |
| Storage | 25° C./97% RH | XRPD pattern unchanged after 11 days |
| | | Purity: 99.9% |
| | 40° C./75% RH | XRPD pattern unchanged after 11 days |
| | | Purity: 99.7% |

| | |
|---|---|
| PLM/SEM | Agglomerates of irregular crystallites, some laths present |

2,4-Dihydroxybenzoic acid (2,4-DHBA Pattern 1)

### Procedure

API (500 mg) was dissolved in 5 vols (2.5 mL) of toluene at 50° C. with stirring at 400 rpm. To the solution, a 1.1 mol. equivalent of the coformer, 2,4 DHBA, (1M stock solution in THF) was added. Following addition of the coformer to the API the solution remained clear. A cooling ramp was applied using a Polar Bear from 50° C. to 5° C. at 0.1° C./min. The solution at 5° C. remained clear.

Maintaining the temperature at 5° C. and stirring rate of 400 rpm, the solution was subjected to anti-solvent addition (n-heptane). A 1:1 solvent/anti-solvent (v/v) ratio was added and although an initial precipitate was observed this redissolved quickly. Before adding the second volume equivalent of anti-solvent, seeds from a previous batch of the cocrystal were added (ARP-1725-42-08). The solution immediately became turbid after the second equivalent of anti-solvent was added. After 2 hours a white suspension had formed. An aliquot was removed, filtered under positive pressure and analysed by XRPD (ARP-1725-71-01A).

This analysis revealed that the solid was a mixture of the cocrystal and API free form. The sample was left stirring at 5° C. over the weekend and re-analysed (ARP-1725-71-01B). As the material was still a mixture the temperature of the solution was raised to 25° C. and after 2 hours a second aliquot was removed and analysed by XRPD (ARP-1725-71-01C). This was still a mixture so an additional 1 volume equivalent of toluene was added at ambient temperature (ie. equivalent volumes of toluene and n-heptane ended up being used). The sample was left stirring at ambient for ca. 17 hours and when re-analysed by XRPD, the diffractogram showed full conversion to 2,4 DHBA Pattern 1 (ARP-1725-71-O1D). The sample was filtered under vacuum and the resulting solid material placed in a vial and dried in a vacuum oven at 40° C. for two hours (Final sample ID: ARP-1725-71-01F). An XRPD was recorded after filtering and prior to drying as well. The sample ID for this was ARP-1725-71-01E.

### Results and Discussion

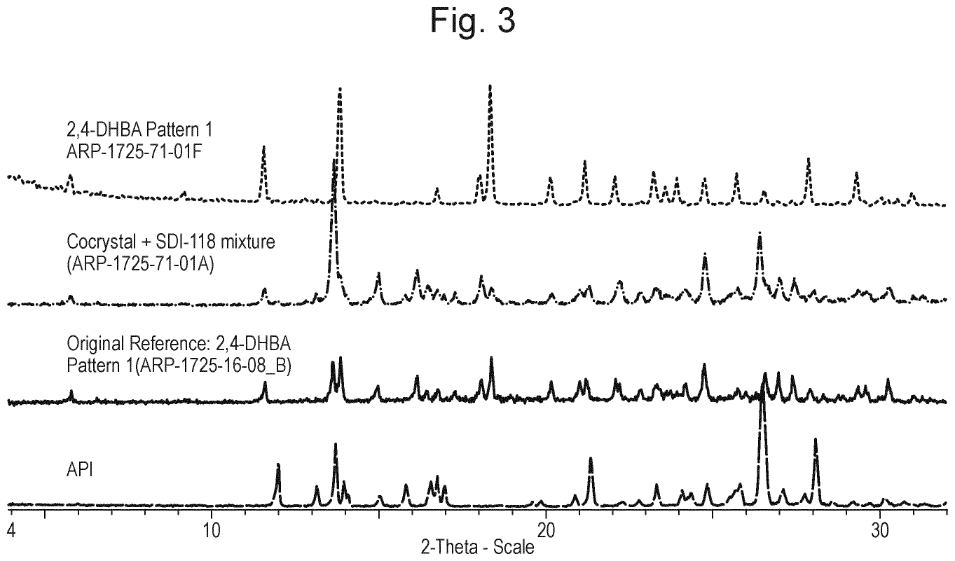

2,54-DHBA Pattern 1 was successfully obtained (FIG. 3) and fully characterised with the results summarised in Table 32. The bis cocrystal form shows good solid-sate properties, i.e. it is anhydrous, has high purity, is only slightly hygroscopic and remains stable to increased temperature and humidity. Some slight changes were noted post GVS in the XRPD this may be due to preferred orientation. A single crystal structure would help to confirm this. The melting point, 138.2° C., is a significant improvement when compared to that of API, which is 85.3° C. The initial small scale reference sample (ARP-1725-16-08_B) did not seem to be phase pure, hence the additional endotherm seen in the DSC of this sample (Table 27).

TABLE 32
Summary of characterisation results for cocrystal with 2,4-dihydroxybenzoic acid
2,4-DHBA Pattern 1 (ARP-1725-71-01F)

| Form | | 2,4-dihydroxybenzoic acid (2,4-DHBA) cocrystal |
|---|---|---|
| Method preparation | | Slow cooling in toluene followed by n-heptane anti- |
| | | solvent additions and then a further 1 equiv. volume |
| | | toluene addition. |
| XRPD | | 2,4-DHBA Pattern 1 |
| HPLC purity | | 99.8% |
| ¹H-NMR | | Consistent with structure |
| | | 2 mol. eq 2,4-DHBA |
| TGA | | No significant mass loss prior to dissociation/ |
| | | degradation then 43.4% w/w loss followed by continual |
| | | decomposition |
| | | (calc. 2 mol. 2,4-DHBA 48.2%) |
| DSC | | Endotherm, onset temp: 138.2° C. (91 J/g) |
| | | Followed by immediate exothermic event |
| VT-XRPD | | XRPD pattern remained unchanged until the sample |
| | | melted at ca. 139° C. |
| GVS | | 0.53% w/w reversible water uptake between 0-90% |
| | | RH. |
| | | Some changes by XRPD post-GVS |
| | | Slightly hygroscopic |
| Storage | 25° C./97% RH | XRPD pattern unchanged after 7 days |
| | | Purity: 99.7% (after ca 1 month on storage) |
| | 40° C./75% RH | XRPD pattern unchanged after 7 days |
| | | Purity: 97.9% (after ca 1 month on storage) |

| | |
|---|---|
| PLM/SEM | Aggregates of fine needles |

3,5-Dihydroxybenzoic acid (3,5-DHBA Pattern 1)

### Procedure

API (500 mg) was dissolved in 5 volumes (2.5 mL) of MIBK at 50° C. with stirring at 400 rpm. To the solution, a 1.1 mol equivalent of the coformer 3,5-DHBA (2 M stock solution in THF) was added. The solution remained clear after addition of the coformer. A cooling ramp from 50° C. to 5° C. at 0.1° C./minute was applied. A clear solution was still present at 5° C.

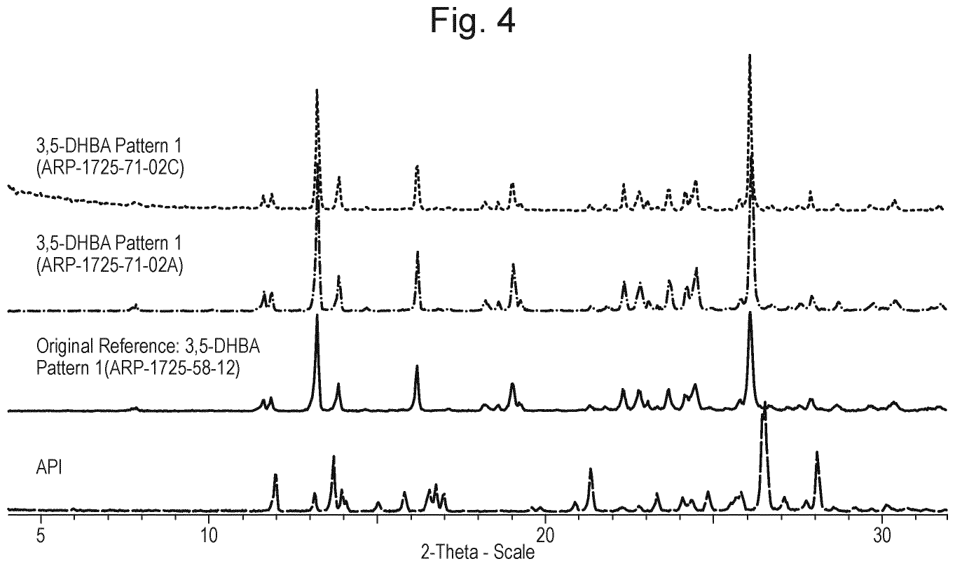

At 5° C. a 1:1 solvent/anti-solvent (n-heptane) volume equivalent was added. The solution showed some initial precipitant but this re-dissolved. The solution was then seeded with a previous cocrystal sample (ARP-1725-58-12), before the second anti-solvent volume equivalent was added. The solution became turbid immediately and after a couple of hours showed a white suspension. After three hours an aliquot was removed from the vial and analysed by XRPD, which showed successful formation of the 3,5-DHBA cocrystal (FIG. 4, ARP-1725-71-02A). To improve the yield, the sample was left at 5° C. over the weekend. The suspension was filtered under vacuum and the resulting solid material placed in a vial and dried in a vacuum oven at 40° C. for two hours. The final sample ID for this material is ARP-1725-71-02C.

### Results and Discussion

The 3,5-DHBA Pattern 1 was successfully obtained (FIG. 4) and fully characterised with the results summarised in Table 33. The mono cocrystal form shows good solid-sate properties, i.e. it is anhydrous, has high purity, is only slightly hygroscopic and remains stable to increased temperature and humidity. The increase in melting point, 113.3° C., is a notable improvement when compared to that of API, which is 85.3° C.

TABLE 33
Summary of characterisation results for cocrystal
with 3,5-dihydroxybenzoic acid
3,5-DHBA Pattern 1 (ARP-1725-71-02C)

| Form | | 3,5-Dihydroxybenzoic Cocrystal |
|---|---|---|
| Coformer | | 3,5-dihydroxybenzoic acid (3,5-DHBA) |
| Method preparation | | Slow cooling in MIBK followed by |
| | | n-heptane anti-solvent additions |
| XRPD | | 3,5-DHBA Pattern 1 |
| HPLC purity | | 99.9% |
| ¹H-NMR | | Consistent with structure |
| | | ~1.1 mol. eq 3,5-DHBA |
| TGA | | Onset of dissociation/decomposition |
| | | ca. 120° C. |
| DSC | | Endotherm, onset temperature: |
| | | 113.3° C. (85 J/g) |
| | | Followed by a broad exotherm |
| VT-XRPD | | XRPD pattern remained unchanged until |
| | | it melted at ca. 113° C. |
| GVS | | ~0.2% w/w reversible water uptake |
| | | between 0-90% RH Majority of uptake |
| | | between 70-90% RH. |
| | | Some hysteresis on desorption. |
| | | Unchanged by XRPD post-GVS |
| | | Slightly hygroscopic |
| Storage | 25° C./97% RH | XRPD Pattern unchanged after 7 days |
| | | Purity: 99.9% |
| | 40° C./75% RH | XRPD Pattern unchanged after 7 days |
| | | Purity: 99.7% |

| | |
|---|---|
| PLM/SEM | Agglomerates of irregular crystallites |

### SUMMARY

All three cocrystals were successfully scaled up using solution-based methods and a summary of the cocrystals and the free form can be found in Table 34. The 2,4 DHBA and 3,5-DHBA cocrystals have good solid state properties including an increase in the melting point by ca. 53 and 28° C. respectively. A notable difference between the 2,4-DHBA cocrystal and the 3,5-DHBA crystal is the 1:2 and 1:1 API to coformer ratio of the two cocrystals. A 1:2 ratio may be more favourable if the therapeutic API dosage is exceptionally low, while a 1:1 ratio would be beneficial if the therapeutic dosage is high. Handling small amounts of the active ingredient can be made easier by producing a final product that requires two equivalents of a coformer.

### CONCLUSIONS

In examples 1 to 3, the propensity for API to form cocrystals with a diverse selection of coformers was investigated.

Initially two cocrystal screens (Screen 1 and 2) with 18 coformers were carried out that included the use of mechanochemistry (dry grinding and liquid-assisted grinding), maturation, cooling, evaporation, sonication and anti-solvent addition. Screen 3 and 4 was performed with an additional set of 18 cocrystals and included similar methods to that of Screen 1 and 2, however, fewer grinding experiments were performed and more solution-based methods were used. From the screening stage, those samples that showed formation of a potential cocrystal being formed (i.e. new peaks in their XRPD patterns) were then characterised using 1H NMR, DSC, TGA, and by evaluating their stability by XRPD after 7 days of being stored at 40° C./75% RH.

Three cocrystals were successfully prepared during the screening stage with 4-HBA, 2,4-DHBA and 3,5-DHBA. All three cocrystals were initially produced using liquid assisted grinding methods, however the 4-HBA cocrystal was also prepared through evaporative methods during the screening phase. The 2,4-DHBA cocrystal was also identified from various solvents during a solvent and cocrystal formation assessment. The three cocrystals were characterised by 1H-NMR, TGA, DSC and by XRPD following storage at 40° C./75% RH and two were found to have significantly higher melting points than Form 1 of API. All three cocrystal were also stable at elevated temperatures and increased humidity levels.

In order to characterise these forms further, all three were selected for scale up. The scale up for the 2,4-DHBA and 3,5-DHBA cocrystals was performed on the 500 mg scale using slow cooling and anti-solvent addition methods to obtain the cocrystals. The 4-HBA cocrystal was prepared on the 100 mg scale using 5 different solvents. Three of the solvents (IPAC, MIBK and Toluene) proved to be effective after following up with an anti-solvent addition (n heptane) step.

The increase in the melting points for the 2,4-DHBA cocrystal (138.2° C.) and the 3,5-DHBA cocrystal (113.3° C.) are a substantial improvement over that of the free form of API (85.3° C.). Both these cocrystals also had good solid-state properties suitable for further development. Of particular interest, is the 1:2 API:coformer ratio for the 2,4-DHBA cocrystal. This could be advantageous from a formulation perspective depending of on the therapeutic dosage of the API. This particular cocrystal also has melting point more than 50° C. higher than that of the free form, thus making it worth considering for development. Based on the solid state characterisation the 4-HBA Pattern 1, 2,4-DHBA Pattern 1 and 3,5-DHBA Pattern 1 will be denoted as 4-HBA Form 1, 2,4-DHBA Form 1 and 3,5-DHBA form 1.

TABLE 34
Summary of the main physical properties for the free form API and the three cocrystals reported.

| Sample ID | API | ARP-1725-77-01B | ARP-1725-71-01F | ARP-1725-71 -02C |
|---|---|---|---|---|
| Coformer | None | 4-hydroxybenzoic acid | 2,4-dihydroxybenzoic acid | 3,5-dihydroxybenzoic acid |
| | | (4-HBA) | (2,4-DHBA) | (3,5-DHBA) |
| API: Coformer ratio | N/A | 1:0.85 | 1:2 | 1:1 |
| Melting point (onset) | 85.3° C. | 87.7° C.* | 138.2° C. | 113.3° C. |
| Purity of API | 99.5% | 99.7% | 99.8% | 99.9% |
| Stability at 25° C./97% RH | Stable, | Stable, | Stable, | Stable, |
| and 40° C./75% RH | XRPD unchanged | XRPD unchanged | XRPD unchanged | XRPD unchanged- |

*multiple thermal events

### Crystal Structure Determination of Three Cocrystals of API

The single crystal X-ray structures of API with three hydroxybenzoic acid coformers were determined at 100 K.

A single crystal of the API 4 hydroxybenzoic acid (4-HBA) 1:1 cocrystal (structure code: PHX-19-095) was obtained by evaporation. The 4-HBA co-crystal crystallises in a triclinic space group, P1, with the final R1[I>2σ(I)]=3.63%.

The single crystals of the API 2,4-dihydroxybenzoic acid (2,4-DHBA) 1:2 cocrystal and API 3,5-dihydroxybenzoic acid (3,5-DHBA) 1:1 cocrystal were both obtained using vapour diffusion. The 2,4-DHBA cocrystal (structure code: PHX-19-093) crystallised in a 1:2 API to coformer ratio and the crystal is monoclinic, space group C2 with the final R1[I>2 σ (I)]=5.22%. The trifluorophenyl group is positionally disordered over two sites in a ratio of 78:22.

The 3,5-DHBA cocrystal (structure code: PHX-19-091) crystallises in a 1:1 API/coformer ratio and is triclinic, space group P1 with the final R1[I>2 σ (I)]=3.14%.

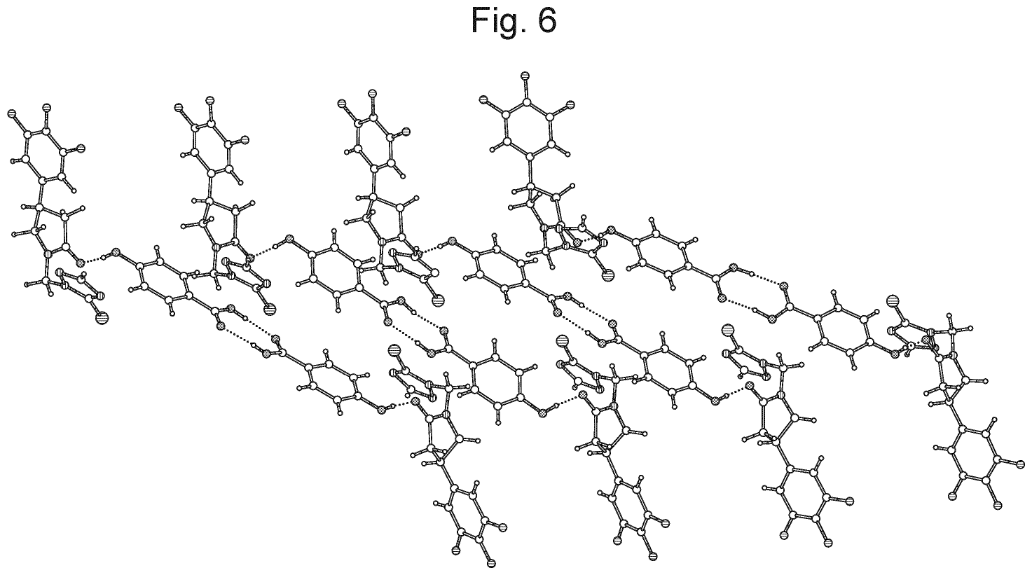

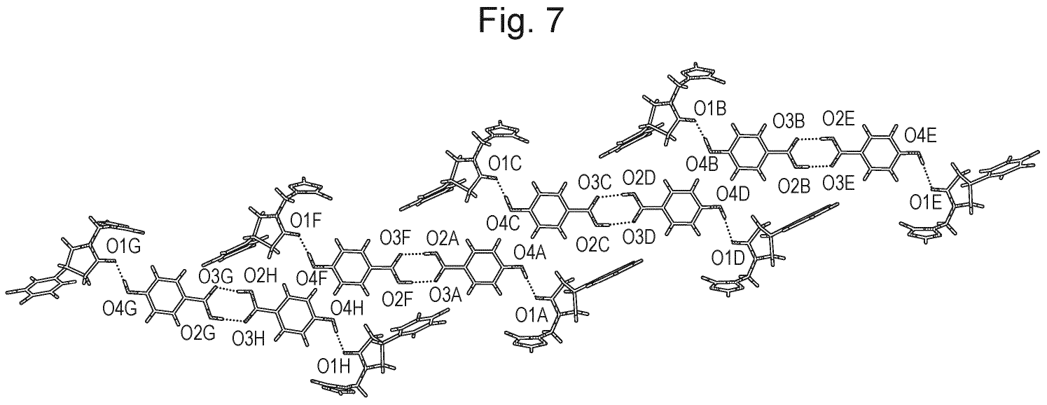

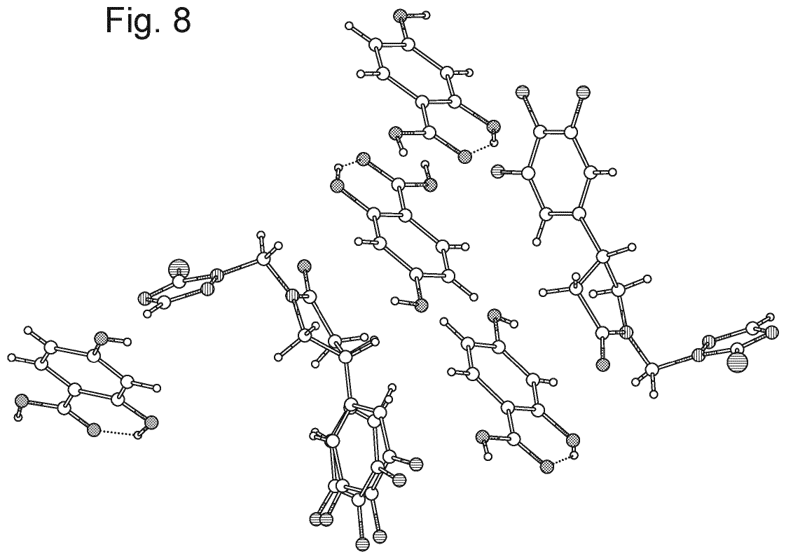

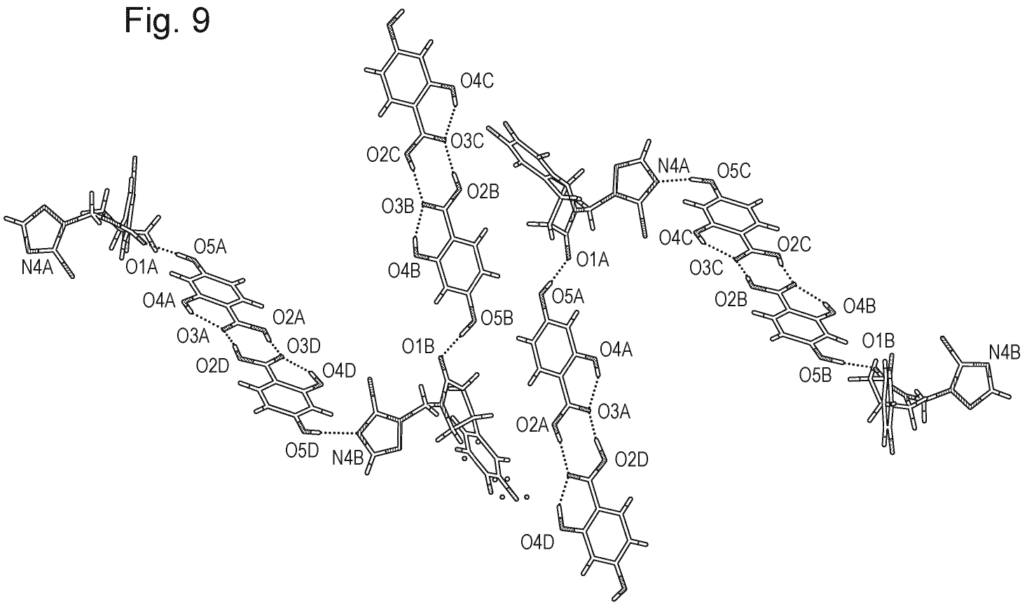

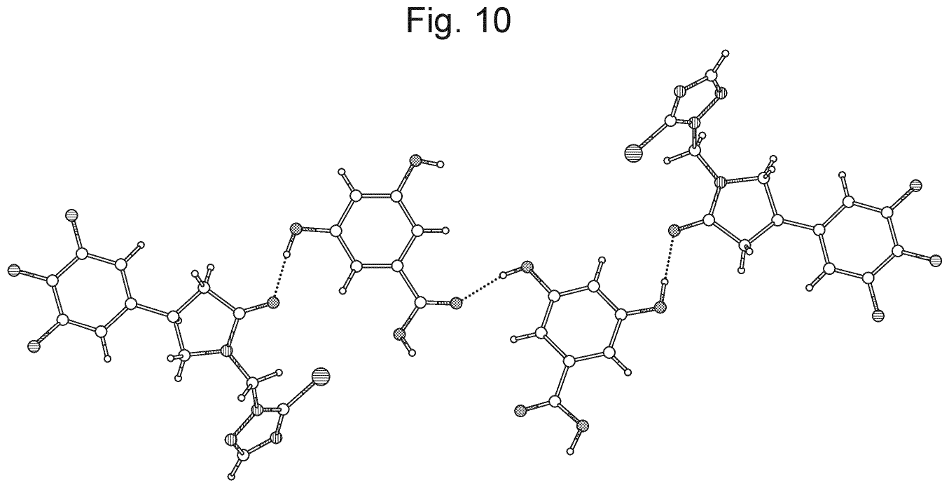

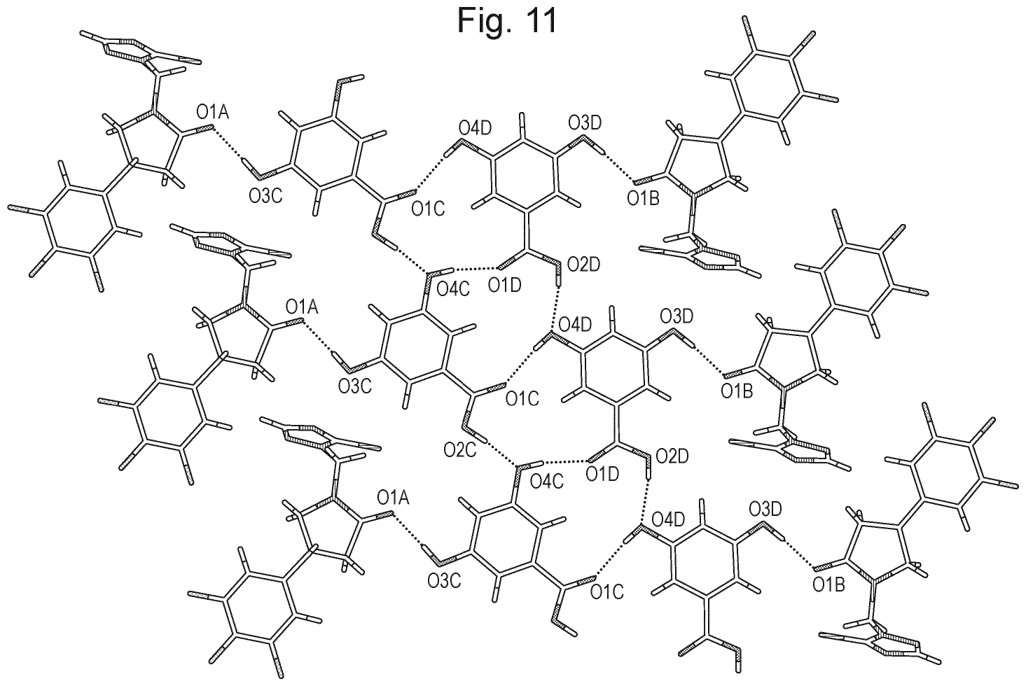

The absolute stereochemistry of the API molecules has been determined for the one stereocentre as being in the R configuration for all crystal structures. The Flack parameters are also all close to zero, therefore confirming the stereochemistry for each structure. Ball and stick representations of the three cocrystals are shown in FIGS. 6, 8 and 10. No proton transfer was observed in the three single crystal structures confirming that all are cocrystals of the API. The hydrogen bonding networks of the three cocrystals are shown in FIGS. 7, 9 and 11.

The simulated XRPD patterns from the single crystal X-ray structures (data collected at 100 K) are consistent with the experimental XRPD patterns that were recorded for the bulk material (collected at room temperature), which confirms that the single crystal structure is representative of the new cocrystals prepared on the small and larger scale. Slight differences in the simulated and experimental diffractograms are most likely attributable to lattice variations with temperature and preferred orientation. A single crystal structure determination at room temperature could help to confirm this.

1. A cocrystal comprising a substituted 2-oxo-1-pyrrolidinyl triazole compound of formula (I): 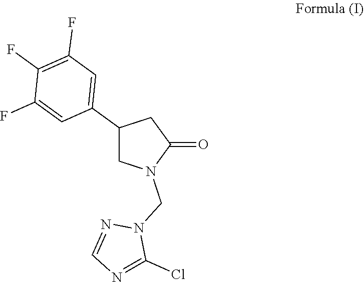 [SMILES: O=C1CC(c2cc(F)c(F)c(F)c2)CN1Cn1ncnc1Cl]
or an isomer thereof, and a carboxylic acid of formula (II): 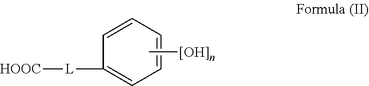
wherein n is from 1 to 5, and
wherein L represents a bond or a C₁₋₄ alkylene group which is unsubstituted or substituted with one, two or three substituents selected from NH₂, OH, SH, fluorine or COOH.

2. A cocrystal according to claim 1, wherein n is from 1 to 3.

3. A cocrystal according to claim 1, wherein L represents a bond.

4. A cocrystal according to claim 1, wherein the carboxylic acid of formula (II) is a hydroxybenzoic acid, a dihydroxybenzoic acid or a trihydroxybenzoic acid.

5. A cocrystal according to claim 1, wherein the carboxylic acid of formula (II) is selected from the group consisting of 4-hydroxybenzoic acid, 2,4-dihydroxybenzoic acid and 3,5-dihydroxybenzoic acid.

6. A cocrystal according to claim 1, wherein L represents a C₁₋₂ alkylene group which is unsubstituted or substituted with NH₂.

7. A cocrystal according to claim 1 wherein the substituted 2-oxo-1-pyrrolidinyl triazole of formula (I) is (4R)-1-[(5-chloro-1H-1,2,4-triazol-1-yl)methyl]-4-(3,4,5-trifluorophenyl)pyrrolidin-2-one.

8. A cocrystal according to claim 1, wherein the cocrystal comprises (4R)-1-[(5-chloro-1H-1,2,4-triazol-1-yl)methyl]-4-(3,4,5-trifluorophenyl)pyrrolidin-2-one and 4-hydroxybenzoic acid.

9. A cocrystal according to claim 1, wherein the cocrystal comprises (4R)-1-[(5-chloro-1H-1,2,4-triazol-1-yl)methyl]-4-(3,4,5-trifluorophenyl)pyrrolidin-2-one and 2,4-dihydroxybenzoic acid.

10. A cocrystal according to claim 1, wherein the cocrystal comprises (4R)-1-[(5-chloro-1H-1,2,4-triazol-1-yl)methyl]-4-(3,4,5-trifluorophenyl)pyrrolidin-2-one and 3,5-dihydroxybenzoic acid.

11. A cocrystal according to claim 1, wherein the cocrystal comprises (4R)-1-[(5-chloro-1H-1,2,4-triazol-1-yl)methyl]-4-(3,4,5-trifluorophenyl)pyrrolidin-2-one and tyrosine.

12. A cocrystal according to claim 1, wherein the melting point of the cocrystal is at least 100° C.

13. A cocrystal according to claim 1, wherein the molar ratio of the substituted 2-oxo-1-pyrrolidinyl triazole of formula (I) to the carboxylic acid of formula (II) is from 3:1 to 1:3.

14. (canceled)

15. (canceled)
